(19) United States
(12) Patent Application Publication
(10) Pub. No.: US 2026/0132369 A1
(43) Pub. Date: May 14, 2026

(54) DEVICE FOR ACQUIRING IMAGES OF SAMPLES OF BIOLOGICAL MATERIAL AND ASSOCIATED METHOD

(71) Applicant: COPAN ITALIA S.P..A, Brescia (IT)

(72) Inventors: Davide Dabellani, Brescia (IT); Stefano Gretter, Brescia (IT); Andrea Mazzolari, Brescia (IT)

(21) Appl. No.: 19/113,394

(22) PCT Filed: Sep. 18, 2023
(86) PCT No.: PCT/IB2023/059229

§ 371 Date: Mar. 19, 2025

(30) Foreign Application Priority Data
Sep. 19, 2022 (IT) 102022000019200

(51) Int. Cl. C12M 1/00; C12M 1/36; H04N 23/51; H04N 23/90

(52) U.S. Cl. CPC C12M 41/14; C12M 29/04; C12M 29/24; C12M 41/48; H04N 23/51; H04N 23/90

(57) ABSTRACT

Device (1) for observing and for acquiring images of samples of biological material (4) comprising: a supporting element (2, 2a, 2b) configured for housing a support (3) for samples of biological material (4); a first camera (6) configured for framing a portion of the supporting element (2, 2a, 2b) and a predefined portion of the support (3); a casing, defining a closed space (100); a first camera (6); a first air conditioner (200), or an inlet (101) for conditioned air arranged on said casing and connected with a second air conditioner (300). The first air conditioner (200) and/or said inlet (101) is configured for conditioning the air contained in the first space (100) for causing a maintenance of a predefined range of values of temperature (T) and humidity (H).

### FIELD OF THE ART

The present disclosure concerns the optical and electronic devices for acquiring images of samples of biological material. The present disclosure also concerns devices and plants for incubating samples of biological material.

### KNOWN ART

There are known devices for showing samples of biological material comprising a camera and a supporting element destined to sustain supports for samples of biological or microbiological material. These supports for samples of biological or microbiological material, in particular, can comprise Petri dishes partially filled with a culture medium. Such devices are destined to take pictures of at least one portion of said container in order to detect the presence of microbiological colonies, typically bacterial, in the culture medium. Such devices may be actuated in a fully manual way or in a more or less automated way. Substantially automated devices of the latest generation, at the time of writing of the present document, are produced for example by the applicant Copan Italia Spa (Pharmalab), by Interscience (Scanstation) and by Rapid Microbiosystem (Growth Direct).

Some devices of known type comprise linear cameras, while others frame one or more images of the entire container and thus of the entire portion of the culture medium.

EP 2 184 346 B1 discloses a device for showing samples of biological material destined to detect micro colonies of size less than 50 μm in at least two orthogonal dimensions, and to quantify the number of micro colonies in a detection area. EP 2 184 346 B1 discloses a unique camera angled illumination system, conceived for optimizing the collection efficiency and for avoiding the obstruction of the incident beam through the collection lens.

U.S. Pat. No. 9,726,602 B2 and EP 2 912 436 B1 show a device and a method for observing samples of biological material on a culture medium. The method provides for directing a light beam onto a portion of a translucid face of a container for samples of biological material in order to detect an illuminated region and a non-illuminated region. The method comprises the acquisition of an image of a portion of the culture medium, wherein said portion is illuminated by the light beam; the acquisition occurs in correspondence of a non-illuminated area of said translucid band, and occurs at a non-zero angle with respect to the direction of propagation of the light beam. In particular, the face of the container for samples of biological material is made translucid by means of a deposit of material diffusing the optical radiation, in particular condensation droplets, in particular water vapour condensation droplets.

US 2017/0044588 A1 discloses a method and an apparatus for detecting micro colonies growing on a culture medium. A sample is irradiated with a light beam, which is incident on the sample with an angle β with respect to the normal direction of the culture medium. An optical receiver receives a reflected, scattered and/or diffused light beam with a different angle α with respect to the angle β.

The devices for acquiring images of samples of biological material often operate on samples of biological material coming from incubators. Incubators for samples of biological material are known to maintain for a predetermined period of time optimal conditions of temperature and humidity for the growth of microbiological samples such as bacteria, fungi, protozoa.

These conditions of optimal temperature and humidity are typically characterized by a temperature above the ambient one, typically in the range of 30-40° C., and are also characterized by a particularly high relative humidity; the relative humidity can reach 90%, for example.

Supports for samples of biological material, taken outside the above-mentioned conditions, are often subject to condensation.

Condensation occurs on surfaces, typically on the surfaces of the sample of biological material, on the culture medium or on the support for the sample of biological material. This condensation can be of the film or droplet type. In the case of film condensation, the vapour condenses for example on the surface of the support, creating a film of liquid. Droplet condensation determines the formation of small droplets for example on the surface of the support.

Condensation, in particular water vapour condensation, severely limits the possibility of acquiring precise images of the sample, in particular in cases where the image is acquired through a closed support or in any case through a transparent wall of the support itself. Given the small dimensions of samples of biological material, the possible presence of condensation, in particular the possible presence of water vapour condensation, can severely limit the ability to even determine whether or not a colony of microbiological samples including bacteria, fungi, protozoa has developed. In the case of differential image acquisition, a microbiological sample count can easily be misled in case of presence of condensation, in particular of a condensation of water vapour, leading to false positives or negatives.

According to the different environmental conditions between the incubator and the space in which images of the support and of the sample of biological material are acquired, condensation may occur on the external or inner surface of the support.

It is noted in particular that microbiological analyses for quality control in the pharmacological field shall be carried out under insulated conditions to avoid contaminations of the culture medium on which the biological sample is deposited. This requires that the support for samples of biological material must remain closed during the growth or incubation process, and also in the case of colony observation or monitoring. In other words, the support for samples of biological material must be maintained with the cover appropriately closed and not manipulated.

The purpose of the present disclosure is to describe a device, a system and a method that allow to solve the above-described drawbacks.

### SUMMARY

The object of the present disclosure will now be described with reference to some of its main aspects, which can be combined with each other or with portions of the following detailed description.

According to the present disclosure it is first of all described a device (1) for observing and for acquiring images of samples of biological material (4) comprising:
a supporting element (2, 2a, 2b) configured for housing a support (3) for biological samples of biological material (4);
at least one first camera (6) oriented toward the supporting element (2, 2a, 2b), wherein said first camera (6) is configured for framing at least one portion of the supporting element (2, 2a, 2b) and/or, in use, at least one predefined portion of the support (3) when housed on the supporting element (2, 2a, 2b), from a first point of observation;
a casing, housing therein at least said supporting element (2, 2a, 2b) and defining a first space (100) at least partially confined or delimited or closed;
at least one first camera (6), preferably arranged within said casing;
   the device comprising at least one first air conditioner (200), and/or comprising at least one inlet (101) for conditioned air arranged on said casing and operatively connected with a second air conditioner (300);
   said first air conditioner (200) and/or said inlet (101) for conditioned air being configured for conditioning the air contained within said first space (100) and being configured for causing a variation and/or a maintenance of at least a predefined range of values of at least one between a temperature (T) of the air and humidity (H) of the air contained within said first space (100), optionally configured for causing a variation and/or a maintenance of a predefined value of at least one between a temperature (T) of the air and humidity (H) of the air contained within said first space (100), for causing a reduction of, or for preventing the formation of, a condensation, in particular of a condensation of water vapour, on said at least one among said supporting element (2, 2a, 2b), said support (3) and said at least one first camera (6).

According to a further non-limiting aspect, the device (1) comprises at least one mover (5) configured for carrying at least one support (3) for samples of biological material (4) from a second space different with respect to said first space (100) toward said supporting element (2, 2a, 2b) and/or vice versa.

According to a further non-limiting aspect, said second space is an incubation space of an incubator (500) configured and specifically destined to incubate samples of biological material (4) optionally contained in one or more supports (3).

According to a further non-limiting aspect, said incubator (500) is optionally and specifically destined to be operatively coupled to said device (1) for receiving one or more supports (3) from the mover (5) of the device (1).

According to a further non-limiting aspect, said support (3) is a closable support, having at least an open configuration and a closed configuration, wherein in said closed configuration the support (3) delimits a predefined inner volume.

According to a further non-limiting aspect, the support (3) comprises at least one upper wall, a lower wall opposed with respect to said upper wall and at least one lateral wall joined to said lower wall.

According to a further non-limiting aspect, said support (3) comprises a cover and at least part of the, preferably all of the, upper wall is a wall of said cover.

According to a further non-limiting aspect, the sample of biological material (4) is contained within said predefined inner volume and on said lower wall.

According to a further non-limiting aspect, said device (1) is configured for handling said support (3) in said closed configuration and/or is configured for acquiring at least one image of the sample of biological material (4) with said support (3) in said closed configuration.

According to a further non-limiting aspect, the device (1) is configured for acquiring at least one image of the sample of biological material (4) through said cover.

According to a further non-limiting aspect, the device (1) is configured for activating said at least one first camera (6) after the arrangement of the support (3) for samples of biological material (4) on said supporting element (2, 2a, 2b).

According to a further non-limiting aspect, the device (1) is configured for activating said at least one first camera (6) after an activation of said at least one first air conditioner (200) and/or of said second air conditioner (300).

According to a further non-limiting aspect, at least one between said upper wall, said lower wall, said at least one lateral wall and/or said cover is at least partially, preferably fully, transparent.

According to a further non-limiting aspect, the device (1) comprises, or is operatively connected with, a data processing unit (400), operatively connected with an incubator (500) configured and specifically destined to incubate samples of biological material (4).

According to a further non-limiting aspect, the incubator (500) is configured and specifically destined to incubate samples of biological material (4) within an incubation space (502) at least partially confined or delimited or closed, optionally contained in one or more supports (3).

According to a further non-limiting aspect, said incubator (500) is configured and specifically destined to be operatively coupled to said device (1) for receiving and/or supplying one or more supports (3) from/to a mover (5) of the device (1).

According to a further non-limiting aspect, the data processing unit (400) is configured for receiving and/or transmitting, optionally automatically, air conditioning electronic data from the incubator (500) and/or toward the incubator (500), or is configured for receiving and/or transmitting, optionally automatically, air conditioning electronic data from the incubator (500) and/or toward the incubator (500) and from said first air conditioner (200) and/or to said first air conditioner (200).

According to a further non-limiting aspect, the data processing unit (400) is in common to said device (1) and to said incubator (500).

According to a further non-limiting aspect, the data processing unit (400) is configured for setting said predefined range of values of at least one between a temperature (T) of the air and humidity (H) of the air contained within said first space (100), optionally said predefined value of at least one between a temperature (T) of the air and humidity (H) of the air contained within said first space (100), on said first and/or second air conditioner (200, 300), according to said electronic data, preferably in such a way that said range of values of at least one between a temperature (T) of the air and humidity (H) of the air contained within said first space (100), optionally said predefined value of at least one between a temperature (T) of the air and humidity (H) of the air contained within said first space (100), is related or equal to range of values of at least one between a temperature (T) of the air and humidity (H) of the air contained within the incubation space (502) of said incubator (500), optionally related or equal to a predefined value of at least one between a temperature (T) of the air and humidity (H) of the air contained within said incubation space (502) of said incubator (500).

According to a further non-limiting aspect, the data processing unit (400) is configured for receiving, optionally automatically, air conditioning electronic data from an incubation data processing unit (511), of said incubator (500).

According to a further non-limiting aspect, said predefined range of values of at least one between a temperature (T) of the air and humidity (H) of the air contained within said first space (100) is related to said range of values of at least one between a temperature (T) of the air and humidity (H) of the air contained within the incubation space (502) of said incubator (500) in such a way that at least a sample value of said predefined range of at least one between a temperature (T) of the air and humidity (H) of the air contained within said first space (100) is out of the range of values of at least one between a temperature (T) of the air and humidity (H) of the air contained within the incubation space (502) of said incubator (500) and/or is in use maintained said reduction of, or is in use prevented the formation of, said condensation, in particular of a condensation of water vapour, on said at least one among said supporting element (2, 2a, 2b), said support (3) and said at least one first camera (6).

According to a further non-limiting aspect, said predefined value of at least one between a temperature (T) of the air and humidity (H) of the air contained within said first space (100) is related to said predefined value of at least one between a temperature (T) of the air and humidity (H) of the air contained within said incubation space (502) of said incubator (500) such as to lie at a predefined difference of said at least between a temperature (T) of the air and humidity (H) of the air with respect to the at least one between a temperature (T) of the air and humidity (H) of the air contained within the incubation space (502) of said incubator (500) and/or allows to maintain said reduction of, or allows to prevent the formation of, said condensation, in particular of said condensation of water vapour, on said at least one among said supporting element (2, 2a, 2b), said support (3) and said at least one first camera (6).

According to a further non-limiting aspect, at least one between said first air conditioner (200) and said second air conditioner (300) is configured for generating a free airflow within said first space (100).

According to a further non-limiting aspect, said free airflow is directed at least partially along a direction concordant with a direction of thermal convection within said first space (100).

According to a further non-limiting aspect, the device comprises:
at least one fan (23) comprising an inlet and an outlet and configured for forcing, in correspondence of said outlet, an airflow within said first space (100);
at least one guide (24, 25, 26) configured for guiding said airflow toward at least one between said supporting element (2, 2a, 2b), said support (3) and said at least one first camera (6).

According to a further non-limiting aspect, the device (1) is specifically configured for determining a simultaneous activation of said at least one fan (23) with said first air conditioner (200) and/or with said second air conditioner (300) for causing said reduction of, or for preventing said formation of, a condensation, in particular of a condensation of water vapour, on said at least one among said supporting element (2, 2a, 2b), said support (3) and said at least one first camera (6).

According to a further non-limiting aspect, in said device (1):
said inlet of said at least one fan (23) is configured for sucking a part of the air present within said first space (100) in correspondence of a first portion of said first space (100) and the outlet of said fan (23) is configured for blowing air in correspondence of a second portion of said first space (100) different with respect to the first portion of said first space; and/or
said fan (23) is arranged in at least one predetermined portion, optionally fixed, with respect to said casing, and is preferably arranged in at least one predetermined position, optionally fixed, within said casing and/or in said first space (100),

According to a further non-limiting aspect, at least one between said first portion and said second portion is a substantially upper or top portion of said first space (100).

According to a further non-limiting aspect, said at least one guide (24, 25, 26) comprises at least one between the following elements:
a suction or thrust duct (24), positioned in substantial correspondence of said at least one fan (23), and extending along at least a predefined direction; and
at least one first deflector (25), preferably a first deflector (25) and a second deflector (26).

According to a further non-limiting aspect, said at least one guide (24, 25, 26), preferably said at least one first deflector (25), is configured for determining a variation of an orientation of the airflow generated in use by said fan (23).

According to a further non-limiting aspect, said at least one guide (24, 25, 26) and said at least one fan (23) are configured and arranged in a reciprocal spatial orientation for determining a substantially closed recirculation of air between said inlet and said outlet.

According to a further non-limiting aspect, said at least one first deflector (25) is positioned within said casing and in said substantially upper or top portion of said first space (100).

According to a further non-limiting aspect, said second deflector (26) is positioned within said casing and in a substantially lower or bottom portion of said first space (100).

According to a further non-limiting aspect, said casing comprises an upper wall, a lower wall opposed with respect to the upper wall and at least one lateral wall having a first and a second portion, the first portion being joined to the upper wall, the second portion being joined to the lower wall.

According to a further non-limiting aspect, the upper wall of the casing is a head wall and/or the lower wall of the casing is a base wall.

According to a further non-limiting aspect, at least one between said first and said second portion are ending portions.

According to a further non-limiting aspect, said casing defines at least one first corner portion in correspondence of a zone adjacent to the union between the upper wall and the lateral wall and/or adjacent to said first portion and defines at least one second corner portion in correspondence of a zone adjacent to the union between the lower wall and the lateral wall and/or adjacent to said second portion.

According to a further non-limiting aspect, said first deflector (25) is positioned in said first corner portion and/or said second deflector (26) is positioned in said second corner portion.

According to a further non-limiting aspect, the device (1) comprises at least one first optical radiation source (10) configured for irradiating at least said predefined portion of the support (3) from a first irradiation direction, in such a way that, in use, at least one portion of the sample of biological material is irradiated through the optical radiation of the first optical radiation source (10).

According to a further non-limiting aspect, said first or second air conditioner (200, 300) is configured for causing a reduction of, or for preventing the formation of, a condensation, in particular of a condensation of water vapour, on said at least one among said supporting element (2, 2a, 2b), said support (3) and said at least one first optical radiation source (10).

According to a further non-limiting aspect, the device (1) comprises a filter (27) for the air contained in said first space (100); the filter (27) is connected on the inlet of said fan (23) and/or to at least one between said first air conditioner (200) and said second air conditioner (300), and is optionally configured for filtering the air before it arrives on the outlet of said fan (23).

According to a further non-limiting aspect, the filter (27) is a filter comprising at least one layer, preferably a plurality of layers, and is optionally a filter destined to filter volatile organic compounds, dusts and, preferably, bacteria or viruses, and/or is an electrostatic filter.

According to a further non-limiting aspect, the filter (27) is a HEPA filter.

According to a further non-limiting aspect, at least one between said first air conditioner (200) and said second air conditioner (300) comprises a thermal machine and/or a device configured for humidifying and/or drying air.

According to the present disclosure it is also described a method for observing and for acquiring images of samples of biological material (4), comprising:
a step of activation of at least one first air conditioner (200) of a device (1) for observing and for acquiring images of samples of biological material (4) and/or of activation of at least one second air conditioner (300) operatively connected to the device (1) by means of an inlet (101) for conditioned air arranged on a casing of the device (1), for conditioning the air contained in a first space (100), defined and at least partially confined or delimited or closed, by said casing;
a step of arrangement of a support (3) for samples of biological material (4) on a supporting element (2, 2a, 2b) housed within the casing of the device (1) for observing and for acquiring images of samples of biological material (4);
a step of framing of at least one portion of the supporting element (2, 2a, 2b) and/or, in use, of at least one predefined portion of the support (3), when housed on the supporting element (2, 2a, 2b), from a first point of observation, by means of at least one first camera (6) oriented toward the supporting element (2, 2a, 2b);
   wherein the step of activation of said at least one first air conditioner (200) and/or of said at least one second air conditioner (300) determines a variation and/or a maintenance of at least a predefined range of values of at least one between a temperature (T) of the air and humidity (H) of the air contained within said first space (100) and, optionally, determines a variation and/or a maintenance of a predefined value of at least one between a temperature (T) of the air and humidity (H) of the air contained within said first space (100) and causes a reduction of, or prevents the formation of, a condensation, in particular of a condensation of water vapour, on said at least one among said supporting element (2, 2a, 2b), said support (3) and said at least one first camera (6).

According to a further non-limiting aspect, the step of framing follows the step of arrangement of the support (3) for samples of biological material (4) on said supporting element (2, 2a, 2b).

According to a further non-limiting aspect, the step of framing follows the step of activation of said at least one first air conditioner (200) and/or of said second air conditioner (300).

According to a further non-limiting aspect, the method comprises a step of transportation of at least one support (3) for samples of biological material (4) from a second space different with respect to said first space (100) toward said supporting element (2, 2a, 2b) and/or vice versa.

According to a further non-limiting aspect, the step of transportation occurs through a mover (5) of the device (1).

According to a further non-limiting aspect, said second space is incubation space of an incubator (500) configured and specifically destined to incubate samples of biological material (4), optionally contained in one or more supports (3).

According to a further non-limiting aspect, the incubator (500) is configured and specifically destined to be operatively coupled to said device (1) for receiving one or more supports (3) from the mover (5) of the device (1).

According to a further non-limiting aspect, said support (3) is a closable support, having at least an open configuration and a closed configuration, wherein in said closed configuration the support (3) delimits a predefined inner volume.

According to a further non-limiting aspect, the support (3) comprises at least one upper wall, a lower wall opposed with respect to said upper wall and a lateral wall joined to said lower wall.

According to a further non-limiting aspect, said support (3) comprises a cover and at least part of the, preferably all of the, upper wall is a wall of said cover.

According to a further non-limiting aspect, the sample of biological material (4) is contained within said predefined inner volume and on said lower wall.

According to a further non-limiting aspect, the method comprises a handling of the support (3) in said closed configuration, and/or comprises a step of acquisition of at least one image of said sample of biological material (4) with said support (3) in said closed configuration.

According to a further non-limiting aspect, the method comprises a step of acquisition of at least one image of said sample of biological material (4) through said cover.

According to a further non-limiting aspect, the method comprises a step of activation of a data processing unit (400).

According to a further non-limiting aspect, the data processing unit (400) is a data processing unit (400) of the device (1), or is a data processing unit (400) operatively external to the device (1).

According to a further non-limiting aspect, the data processing unit (400) is operatively connected with an incubator (500) configured and specifically destined to incubate samples of biological material (4) within an incubation space (502) of the incubator (500) at least partially confined or delimited or closed, optionally contained in one or more supports (3).

According to a further non-limiting aspect, said incubator (500) is optionally configured and specifically destined to be operatively coupled to said device (1) for receiving one or more supports (3) from a mover of the device (1).

According to a further non-limiting aspect, the method comprises a step of reception and/or of transmission, optionally automatic, through said data processing unit (400), of air conditioning electronic data from the incubator (500) and/or to the incubator (500) and/or comprises a step of reception and/or transmission, optionally automatic, through said data processing unit (400), of air conditioning electronic data from the first air conditioner (200) and/or to said first air conditioner (200).

According to a further non-limiting aspect, the method comprises a step of setting of said predefined range of values of at least one between a temperature (T) of the air and humidity (H) of the air contained within said first space (100), optionally said predefined value of at least one between a temperature (T) of the air and humidity (H) of the air contained within said first space (100), on said first and/or second air conditioner (200, 300), according to said electronic data, preferably in such a way that said range of values of at least one between a temperature (T) of the air and humidity (H) of the air contained within said first space (100), optionally said predefined value of at least one between a temperature (T) of the air and humidity (H) of the air contained within said first space (100), is related or equal to range of values of at least one between a temperature (T) of the air and humidity (H) of the air contained within the incubation space (502) of said incubator (500), optionally related or equal to a predefined value of at least one between a temperature (T) of the air and humidity (H) of the air contained within the incubation space (502) of said incubator (500).

According to a further non-limiting aspect, the method comprises a step of reception, optionally automatic, through the data processing unit (400), of air conditioning electronic data from an incubation data processing unit (511), of said incubator (500).

According to a further non-limiting aspect, the step of activation of said first air conditioner (200) and/or of said second air conditioner (300) determines the generation of a free airflow and, optionally at least by effect of the activation of at least one between said first and said second air conditioner (200, 300), said free airflow is directed at least partially along a direction concordant with a direction of thermal convection within said first space (100).

According to a further non-limiting aspect, the method comprises a step of activation of at least one fan (23) of said device (1) and comprises an inlet and an outlet, for forcing, in correspondence of said outlet, an airflow within said first space (100).

According to a further non-limiting aspect, the method comprises a step of guide, through at least one guide (24, 25, 26) of said device (1), of said airflow toward at least one between said supporting element (2, 2a, 2b), said support (3) and said at least one first camera (6).

According to a further non-limiting aspect, the method comprises a simultaneous execution of the step of activation of said fan (23) with the step of activation of said first air conditioner (200) and/or of said second air conditioner (300) for causing said reduction of, or for preventing the formation of, a condensation, in particular of a condensation of water vapour, on said at least one among said supporting element (2, 2a, 2b), said support (3) and said at least one first camera (6).

According to a further non-limiting aspect, the step of activation of said at least one fan (23) determines a suction, through the inlet, of a part of the air present within said first space (100) from a first portion of said first space (100) and determines a blowing, through said outlet, of air in correspondence of a second portion of said first space (100) different with respect to the first portion of said first space (100).

According to a further non-limiting aspect, the method comprises arranging the fan (23) in at least one predetermined portion, optionally fixed, with respect to said casing, and preferably comprises arranging the fan (23) in at least a predetermined position, optionally fixed, within said casing and/or in said first space (100).

According to a further non-limiting aspect, at least one between said first portion and said second portion is a substantially upper or top portion of said first space (100).

According to a further non-limiting aspect, said at least one guide (24, 25, 26) comprises at least one between the following elements:
a suction or thrust duct (24), positioned in substantial correspondence of said at least one fan (23), and extending along at least a predefined direction; and
at least one first deflector (25), preferably a first deflector (25) and a second deflector (26).

According to a further non-limiting aspect, the step of guiding, through at least one guide (24, 25, 26) of said device (1), of said airflow, comprises a variation of an orientation of the airflow generated through the step of activation of said fan (23).

According to a further non-limiting aspect, the method comprises a substantially closed recirculation of air between said inlet and said outlet, said recirculation being determined by a reciprocal spatial orientation between said at least one guide (24, 25, 26) and said at least one fan (23).

According to a further non-limiting aspect, the method comprises a step of positioning of the at least one first deflector (25) within said casing and in said substantially upper or top portion of said first space (100), and/or comprises a step of positioning of the second deflector (26) within said casing and in a substantially lower or bottom portion of said first space (100).

According to a further non-limiting aspect, said casing comprises an upper wall, a lower wall opposed with respect to the upper wall and at least one lateral wall having a first and a second portion, the first portion being joined to the upper wall, the second portion being joined to the lower wall.

According to a further non-limiting aspect, at least one between said first and said second portion are ending portions.

According to a further non-limiting aspect, said casing defines at least one first corner portion in correspondence of a zone adjacent to the union between the upper wall and the lateral wall and/or adjacent to said first portion and defines at least one second corner portion in correspondence of a zone adjacent to the union between the lower wall and the lateral wall and/or adjacent to said second portion.

According to a further non-limiting aspect, the step of positioning of said first deflector (25) determines an allocation of said first deflector (25) in said first corner position and/or the step of positioning of said second deflector (26) determines an allocation of said second deflector (26) in said second corner portion.

According to a further non-limiting aspect, the method comprises a step of activation of at least one first optical radiation source (10) for irradiating at least said predefined portion of the support (3) from a first irradiation direction, in such a way that, in use, at least one portion of the sample of biological material is irradiated through the optical radiation of the first optical radiation source (10).

According to a further non-limiting aspect, the step of activation of said at least one first air conditioner (200) of said device (1) and/or the step of activation of said at least one second air conditioner (300) causes a reduction of, or prevents the formation of, a condensation, in particular of a condensation of water vapour, on said at least one among said supporting element (2, 2a, 2b), said support (3) and said at least one first optical radiation source (10).

According to a further non-limiting aspect, the method comprises a step of filtering of the air contained in said first space (100) by means of a filter (27), wherein the filter (27) is connected on the inlet of said fan (23) and/or to at least one between said first air conditioner (200) and said second air conditioner (300) and is optionally configured for carrying out a filtering of the air before it arrives on the outlet of said fan (23).

According to a further non-limiting aspect, said step of filtering occurs by means of a HEPA filter (27).

According to the present disclosure it is also described a computer program, comprising portions of software code that when executed by a data processing unit cause the execution of a method for observing and for acquiring images of samples of biological material (4), comprising:
a step of activation of at least one first air conditioner (200) of a device (1) for observing and for acquiring images of samples of biological material (4) and/or of activation of at least one second air conditioner (300) operatively connected to the device (1) by means of an inlet (101) for conditioned air arranged on a casing of the device (1), for conditioning the air contained in a first space (100), defined and at least partially confined or delimited or closed, by said casing;
a step of activation of at least one first mover (5) for carrying out an arrangement of a support (3) for samples of biological material (4) on a supporting element (2, 2a, 2b) housed within the casing of the device (1) for observing and for acquiring images of samples of biological material (4);
a step of framing of at least one portion of the supporting element (2, 2a, 2b) and/or, in use, of at least one predefined portion of the support (3), when housed on the supporting element (2, 2a, 2b), from a first point of observation, by means of at least one first camera (6) oriented toward the supporting element (2, 2a, 2b);
   wherein the step of activation of said at least one first air conditioner (200) and/or of said at least one second air conditioner (300) determines a variation and/or a maintenance of at least a predefined range of values of at least one between a temperature (T) of the air and humidity (H) of the air contained within said first space (100) and, optionally, determines a variation and/or a maintenance of a predefined value of at least one between a temperature (T) of the air and humidity (H) of the air contained within said first space (100) and causes a reduction of, or prevents the formation of, a condensation, in particular of a condensation of water vapour, on said at least one among said supporting element (2, 2a, 2b), said support (3) and said at least one first camera (6).

According to the present disclosure it is also described a memory support, comprising said computer program.

According to the present disclosure it is also described a system comprising a device (1) according to one or more of the aspects described herein and an incubator (500) configured and specifically destined to incubate samples of biological material (4), optionally contained in one or more supports (3) and optionally configured and specifically destined to be operatively coupled to said device (1) for receiving one or more supports (3) from a mover of the device (1),

wherein the device (1) and the incubator (500) are operatively coupled one another for realizing a first space (100) and a second space substantially connected one another and insulated with respect to an external space.

According to the present disclosure it is also described an incubator (500), configured and specifically destined to incubate samples of biological material (4), comprising:
a casing defining an incubation space (502) configured for being substantially insulated from the external space;
at least one first incubation station (505) arranged in said incubation space (502) and configured for housing at least one support (3) and preferably for housing a plurality of supports (3);
   the incubator (500) being configured and specifically destined to be operatively coupled to a device (1) for observing and for acquiring images of samples of biological material (4), preferably according to one or more of the aspects described herein, for receiving one or more supports (3) containing samples of biological material (4) from said device (1) and/or supplying one or more supports (3) containing samples of biological material (4) to said device (1);
   the incubator (500) comprising:
an air conditioner (300), configured for conditioning the air contained within said incubation space (502) and configured for causing a variation and/or a maintenance of at least a predefined range of values of at least one between a temperature (T) of the air and humidity (H) of the air contained within said incubation space (502), optionally configured for causing a variation and/or a maintenance of a predefined value of at least one between a temperature (T) of the air and humidity (H) of the air contained within said incubation space (502), for determining an incubation of at least one sample of biological material (4), wherein the air conditioner (300) of the incubator (500) is configured for operatively synchronizing an air conditioner (200) of said device (1) or operatively associated to the device (1), for making the predefined range of values of at least one between a temperature (T) of the air and humidity (H) of the air contained within said incubation space (502), optionally for giving the variation and/or the maintenance of a predefined value of at least one between a temperature (T) of the air and humidity (H) of the air contained within said incubation space (502), related or equal to the predefined range of values of at least one between a temperature (T) of the air and humidity (H) of the air contained within said first space (100), optionally related or equal to the variation and/or to the maintenance of a predefined value of at least one between a temperature (T) of the air and humidity (H) of the air contained within said first space (100); and/or
at least one opening (510) of transit of supports (3), configured and specifically destined to put said incubation space (502) and a first space (100) defined by a casing of the device (1) in communication for realizing an unique space having a same range of values of at least one between a temperature (T) of the air and humidity (H) of the air, optionally a same predefined value of at least one between a temperature (T) of the air and humidity (H), said unique space being at least partially confined or delimited or closed.

According to a further non-limiting aspect, the incubator (500) is configured for transmitting to, and/or receiving from, said device (1) conditioning electronic data.

According to a further non-limiting aspect, said electronic data are destined to determine a setting of said predefined range of values of at least one between a temperature (T) of the air and humidity (H) of the air contained within said first space (100), optionally of said predefined value of at least one between a temperature (T) of the air and humidity (H) of the air contained within said first space (100), by action of said air conditioner (200) of said device (1) or operatively associated to said device (1), in such a way that said range of values of at least one between a temperature (T) of the air and humidity (H) of the air contained within said first space (100), optionally said predefined value of at least one between a temperature (T) of the air and humidity (H) of the air contained within said first space (100), is related or equal to range of values of at least one between a temperature (T) of the air and humidity (H) of the air contained within the incubation space (502) of said incubator (500), optionally related or equal to a predefined value of at least one between a temperature (T) of the air and humidity (H) of the air contained within said incubation space (502) of said incubator (500).

According to the present disclosure it is also described a method of incubation of samples of biological material (4), comprising:
a step of activation of an air conditioner (300) of an incubator (500), for conditioning the air contained within said incubation space (502) and for causing a variation and/or a maintenance of at least a predefined range of values of at least one between a temperature (T) of the air and humidity (H) of the air contained within said incubation space (502), optionally for causing a variation and/or a maintenance of a predefined value of at least one between a temperature (T) of the air and humidity (H) of the air contained within said incubation space (502), for determining an incubation of at least one sample of biological material (4), and wherein the method comprises a step of operative synchronization between the incubator (500) and an air conditioner (200) of the device (1) or operatively associated to the device (1), for making the predefined range of values of at least one between a temperature (T) of the air and humidity (H) of the air contained within said incubation space (502), optionally for giving the variation and/or the maintenance of a predefined value of at least one between a temperature (T) of the air and humidity (H) of the air contained within said incubation space (502), related or equal to the predefined range of values of at least one between a temperature (T) of the air and humidity (H) of the air contained within said first space (100), optionally related or equal to the variation and/or to the maintenance of a predefined value of at least one between a temperature (T) of the air and humidity (H) of the air contained within said first space (100); and/or
an operative coupling between the incubator (500) and a device (1) for observing and for acquiring images of samples of biological material (4), preferably according to one or more of the aspects described herein, for receiving one or more supports (3) containing samples of biological material (4) or from said device (1) and/or for supplying and/or receiving one or more supports (3) containing samples of biological material (4) to said device (1), wherein the operative coupling comprises the transit of at least one support (3) within at least one opening (510) of the incubator (500) which puts in communication the incubation space (505) with a first space (100) defined by a casing of the device (1) for realizing an unique space having a same range of values of at least one between a temperature (T) of the air and humidity (H) of the air, optionally a same predefined value of at least one between a temperature (T) of the air and humidity (H), said unique space being at least partially confined or delimited or closed;
a step of arrangement of a sample of biological material (4), housed in a respective support (3), within an incubation space (502) of an incubator (500) where is present a first incubation station (505) configured for housing at least one support (3) and preferably for housing a plurality of supports (3).

According to a further non-limiting aspect, the method comprises a transmission of conditioning electronic data from said incubator (500) to said device (1) and/or a reception of conditioning electronic data from said device (1) to said incubator (500).

According to a further non-limiting aspect, at least the transmission of conditioning electronic data to said device (1) determines a setting of said predefined range of values of at least one between a temperature (T) of the air and humidity (H) of the air contained within said first space (100), optionally of said predefined value of at least one between a temperature (T) of the air and humidity (H) of the air contained within said first space (100), by action of a first air conditioner (200) of said device (1) or operatively associated to said device (1), in such a way that said range of values of at least one between a temperature (T) of the air and humidity (H) of the air contained within said first space (100), optionally said predefined value of at least one between a temperature (T) of the air and humidity (H) of the air contained within said first space (100), is equal to a range of values of at least one between a temperature (T) of the air and humidity (H) of the air contained within the incubation space (502) said incubator (500), optionally equal to a predefined value of at least one between a temperature (T) of the air and humidity (H) of the air contained within said incubation space (502) of said incubator (500).

According to the present disclosure it is also described a device (1) for observing and for acquiring images of samples of biological material (4) comprising:
a supporting element (2, 2a, 2b) configured for housing a support (3) for samples of biological material (4);
at least one first camera (6) oriented toward the supporting element (2, 2a, 2b), wherein said first camera (6) is configured for framing at least one portion of the supporting element (2, 2a, 2b) and/or, in use, at least one predefined portion of the support (3) when housed on the supporting element (2, 2a, 2b), from a first point of observation;
a casing, housing therein at least said supporting element (2, 2a, 2b) and defining un first space (100) at least partially confined or delimited or closed;
at least one first camera (6), preferably arranged within said casing;
   the device comprises at least one air conditioning element (200, 101), configured for conditioning, or for allowing to condition, the air contained within said first space (100) and configured for causing a variation and/or a maintenance of at least a predefined range of values of at least one between a temperature (T) of the air and humidity (H) of the air contained within said first space (100), optionally configured for causing a variation and/or a maintenance of a predefined value of at least one between a temperature (T) of the air and humidity (H) of the air contained within said first space (100), for causing a variation of, or for preventing the formation of, a condensation, in particular of a condensation of water vapour, on said at least one among said supporting element (2, 2a, 2b), said support (3) and said at least one first camera (6).

According to a further non-limiting aspect, said at least one air conditioning element (200, 101) comprises at least one first air conditioner (200), and/or at least one inlet (101) for conditioned air arranged on said casing and operatively connected with a second air conditioner (300).

According to a further non-limiting aspect, said second air conditioner (300) is operationally external, and/or is not part of, said device (1).

According to a further non-limiting aspect, said first air conditioner (200) and/or said inlet (101) for conditioned air is configured for conditioning the air contained within said first space (100) and is configured for causing a variation and/or a maintenance of at least a predefined range of values of at least one between a temperature (T) of the air and humidity (H) of the air contained within said first space (100), optionally configured for causing a variation and/or a maintenance of a predefined value of at least one between a temperature (T) of the air and humidity (H) of the air contained within said first space (100), for causing a variation of, or for preventing the formation of, a condensation, in particular of a condensation of water vapour, on said at least one among said supporting element (2, 2a, 2b), said support (3) and said at least one first camera (6).

According to the present disclosure it is also described a method for observing and for acquiring images of samples of biological material (4), comprising:
a step of activation of at least one air conditioning element (200, 101, 300) for conditioning the air contained in a first space (100), defined and at least partially confined or delimited or closed, by said casing;
a step of arrangement of a support (3) for samples of biological material (4) on a supporting element (2, 2a, 2b) housed within the casing of the device (1) for observing and for acquiring images of samples of biological material (4);
a step of framing of at least one portion of the supporting element (2, 2a, 2b) and/or, in use, of at least one predefined portion of the support (3), when housed on the supporting element (2, 2a, 2b), from a first point of observation, by means of at least one first camera (6) oriented toward the supporting element (2, 2a, 2b);
   wherein the step of activation of said at least one air conditioning element (200, 101, 300) determines a variation and/or a maintenance of at least a predefined range of values of at least one between a temperature (T) of the air and humidity (H) of the air contained within said first space (100) and, optionally, determines a variation and/or a maintenance of a predefined value of at least one between a temperature (T) of the air and humidity (H) of the air contained within said first space (100) and causes a reduction of, or prevents the formation of, a condensation, in particular of a condensation of water vapour, on said at least one among said supporting element (2, 2a, 2b), said support (3) and said at least one first camera (6).

According to a further non-limiting aspect, the step of activation of the at least one air conditioning element (200, 101, 300) comprises the activation of at least one first air conditioner (200) of a device (1) for observing and for acquiring images of samples of biological material (4) and/or the activation of at least one second air conditioner (300) operatively connected to the device (1) by means of an inlet (101) for conditioned air arranged on a casing of the device (1).

According to a further non-limiting aspect, the step of activation of said at least one first air conditioner (200) and/or of said at least one second air conditioner (300) determines a variation and/or a maintenance of at least a predefined range of values of at least one between a temperature (T) of the air and humidity (H) of the air contained within said first space (100) and, optionally, determines a variation and/or a maintenance of a predefined value of at least one between a temperature (T) of the air and humidity (H) of the air contained within said first space (100) and causes a reduction of, or prevents the formation of, a condensation, in particular of a condensation of water vapour, on said at least one among said supporting element (2, 2a, 2b), said support (3) and said at least one first camera (6).

### FIGURES

The present disclosure will be described below in some of its preferred but non-limiting embodiments, and with the aid of some figures, which are described below.

FIG. 1 shows a first embodiment of a device for observing samples of biological material; the embodiment of FIG. 1 comprises a single camera for the acquisition of images of one or more of said samples. The embodiment of FIG. 1 comprises an air conditioner operatively connected with an air conditioner of an incubator for incubating supports containing samples of biological material transferred by the device 1.

FIG. 2 shows a second embodiment of a device for observing samples of biological material; the embodiment of FIG. 2 comprises an air conditioner for conditioning a space within the casing of the device 1 so as to reduce the risk of formation of condensation, in particular of a condensation of water vapour.

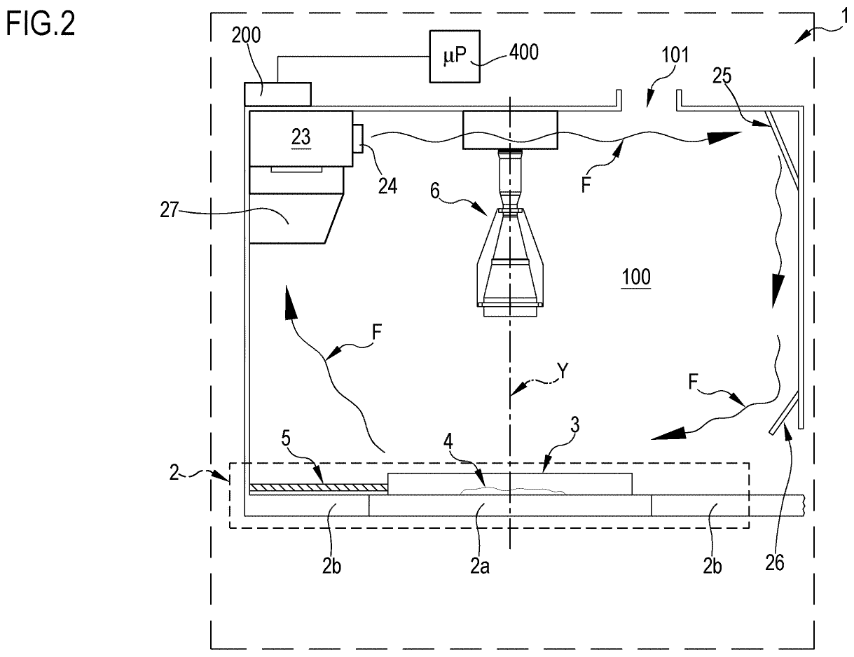

FIG. 3 shows a further embodiment of a device for observing samples of biological material; the embodiment of FIG. 3 comprises a plurality of cameras for the acquisition of images of one or more of said samples.

FIG. 4 and FIG. 5 show respectively a front view and a sectional top view of an incubator configured for being operatively connected with a device for the acquisition of images of samples of biological material contained within one or more supports.

FIG. 6 shows a flow chart of a non-limiting example of a temperature adjustment process of an inner space of the device object of the present disclosure.

### DETAILED DESCRIPTION

With reference number 1 it is indicated as a whole a device for observing and acquiring images of samples of biological material.

For the purposes of the present disclosure, as samples of biological material are intended samples containing any element of biological or microbiological nature, in a non-limiting extent, secretions, organic fluids, one or more microorganism colonies, in particular fungi, preferably molds and yeasts, and/or bacteria colonies and/or protozoa.

The following description will refer to particular configurations of use or steps of a method for observing samples of biological material. Where the description refers to a particular configuration of use, typically it will not be repeated the same description with specific reference to a step of a method, in order not to excessively increase the length of the following detailed description; in the same way, where the description refers to a step of a method, typically it will not be repeated the same description with specific reference to a configuration of use. It is then intended that determined steps can correspond to determined configurations of use and vice versa.

The device 1 herein described comprises a casing which defines an inner space 100; the casing comprises an upper wall and a lower wall, opposed with respect to the upper wall. In particular, the upper wall is a head wall and the lower wall is a bottom wall. One or more lateral walls are connected to the upper wall and to the lower wall in correspondence of a respective first and second portion. The first and the second portion of the lateral walls can be ending portions.

### The Supporting Element and the Support for Biological Samples

The device for observing samples of biological material 1 comprises a supporting element 2, 2a, 2b which is destined and specifically configured for housing at least one support 3 for samples of biological material.

The supporting element 2, 2a, 2b is preferably provided with a substantially planar upper surface that, in use, lies on a substantially horizontal plane. The supporting element 2, 2a, 2b comprises a first portion 2a and a second portion 2b. The first portion 2a is an inner portion with respect to the second portion 2b, which is therefore an external portion.

In a non-limiting embodiment, the supporting element 2, 2a, 2b assumes a substantially discoidal configuration, and has then a substantially circular transversal section; the first portion 2a is a radially inner portion, whereas the second portion 2b is a radially external portion.

In particular, in the attached figures it is represented a specific embodiment of the supporting element in shape of a supporting plate. Where in the present description, a generic reference is made to the supporting element 2, 2a, 2b, it shall be intended that what is described will be in particular valid for a supporting element in shape of a supporting plate.

In a non-limiting embodiment, the first portion 2a is optically transparent and is substantially hollow or provided with an appropriate transparent supporting element, for example a glass or a plastic material.

In an embodiment, the first portion 2a can be provided with an optical diffuser, configured for diffusing the optical radiation along a plurality of directions by reducing the power concentration of the optical radiation along a predefined direction or reduced angle of directions; preferably, this optical diffuser is translucid, or opaline. The optical diffuser can be realized with any material of known type, for example and non-limiting thereto, in glass or plastic material (for example, polycarbonate).

An axis Y departs from the supporting element 2, 2a, 2b in a substantially orthogonal direction with respect to the latter.

The supporting element 2, 2a, 2b is preferably conceived for rotating around a predetermined axis, and this axis is actually the axis Y. In some embodiments, however, this supporting element is configured and specifically destined for linearly translating along at least one direction. The translation along at least one direction occurs in operating association to the rotation or alternatively to the rotation.

In a preferred but non-limiting embodiment, the supporting element 2, 2a, 2b is realized at least partially in metallic material, for example aluminum.

It is observed that the upper surface comprises a first area being part of the first portion 2a, and a second area being part of the second portion 2b. Therefore, each one between the first and the second portion 2a, 2b shows an its own upper surface and these surfaces are substantially coplanar. Preferably, then, there are no steps between the first portion 2a and the second portion 2b.

For the purposes of the present disclosure are defined a first and a second side of the supporting element 2, 2a, 2b. In particular, the first side of the supporting element 2, 2a, 2b is the upper side whereas the second side is the lower side.

The device 1 object of the present disclosure comprises an actuator, in particular an actuator, in particular a motor, configured for moving, in particular for moving, in particular for putting in rotation, the supporting element 2, 2a, 2b. In a non-limiting embodiment, the motor is an electric motor and in particular can be a stepper-type motor, controlled by the data processing unit of the device 1. The motor in use causes a movement, and in particular a rotation of the supporting element 2, 2a, 2b with direct or indirect transmission, for example through a toothed belt.

Said actuator can be in particular configured and specifically destined to allow an always clockwise, or always counterclockwise rotation or to allow an alternatively clockwise and counterclockwise rotation.

In an embodiment, the support 3 for samples of biological material comprises a Petri dish.

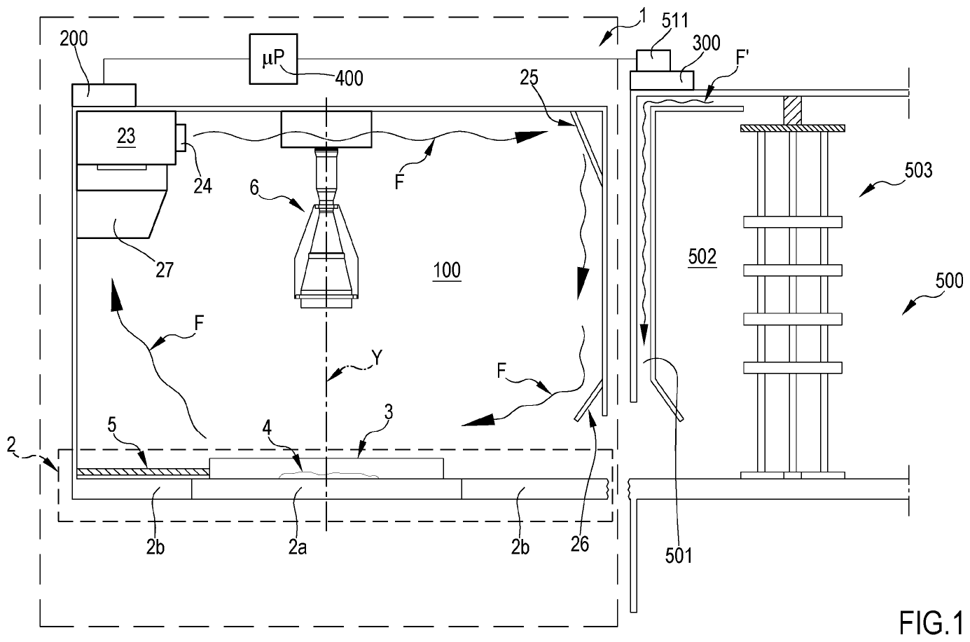

As schematically shown in FIG. 1, in a non-limiting embodiment, the support 3 comprises a bottom portion, upon which in use is deposited a culture medium, which typically contains a sample of biological material 4, at least one lateral wall 3b, which extends in substantially oblique direction with respect to the bottom portion, and a head portion 3a, which extends in substantially oblique direction with respect to the lateral wall 3b.

In an embodiment, the support 3 has a substantially circular section, and therefore the lateral wall 3b is unique and extends seamlessly; in FIG. 1, the lateral wall 3b is orthogonal with respect to the bottom portion of the support 3 and the head portion 3a lies on a plane substantially parallel to the plane upon which lies the bottom portion. The support 3 can for example and in a non-limiting extent have a diameter substantially comprised between 30 mm and 170 mm, or between 40 mm and 160 mm, or between 50 mm and 150 mm.

In a non-limiting embodiment, the head portion 3a is removably connectible to the at least one lateral wall 3b, and therefore realizes a cover which if needed can be removed for gaining access to the culture medium. In an embodiment the head portion 3a, when connected to the lateral wall 3b, substantially seals a cavity of the support 3.

The culture medium comprises one or more solid or liquid solutions containing nutritional substances on which it is possible to grow eukaryotic and prokaryotic cells. A specific and non-limiting embodiment of culture medium is the Agar.

In an alternative embodiment, the support 3 can be substantially planar and open.

The support 3 can be realized in different materials among which plastic materials and/or in glass.

The support 3, especially when shaped so as to define an inner cavity, is preferably realized in an optically substantially transparent material.

In particular, a specific and non-limiting embodiment of the support 3 comprises an upper wall, a lower wall opposed with respect to said upper wall and at least one lateral wall joined to said lower wall.

The support 3 comprises a cover; at least part of the, preferably all of the, upper wall is a wall of the cover. The sample of biological material 4 is in use contained within the predefined inner volume defined by the above-mentioned walls.

Preferably, at least one the above-mentioned walls and/or of the cover is transparent.

In a specific embodiment, the support 3 has a substantially circular plan. The transparent cover detects a perimeter of substantially circular shape. The cover is screwed on the lateral wall of the support 3 or is fixed with a contrasting insertion on the lateral wall of the support 3.

The support 3 can have a reference marking which allows to identify a rotation with respect to a predefined point, precisely detected by said marking. The marking can be an engraving, a raised portion, a sticker, a painted portion. Preferably but non-limiting thereto, the marking is arranged on the lateral wall, and even more preferably on an external side of the lateral wall.

An optically substantially transparent material is a material which allows the passage of an optical radiation, in particular in the visible domain, without appreciable scattering. A substantially transparent material is different from a substantially translucid material, wherein, instead, an optical radiation herein incident is capable of passing, but not necessarily following the Snell's law; the optical radiation, in a translucid material, can be scattered in correspondence of the two interfaces (of input and output of the radiation) or internally to the material itself. A particular expression of translucid materials are opaque materials. These materials cannot substantially be traversed by an optical radiation, which is substantially integrally attenuated by the material itself. In particular are substantially transparent at least the head portion 3a and/or the lateral wall of the support.

It is observed in particular that at least one side of the support 3 in use directed toward the cameras on board of the device 1 is optically substantially transparent.

In configurations of use wherein at least one image of the sample of biological material 4 is acquired, the cover of the support 3 is maintained in contact with the base, and if present, with the lateral wall, of the support itself, to create a protected space for the sample of biological material 4, uncontaminated by the various manipulations to which the support 3 is subjected.

### Devices for Acquiring Images

The device 1 object of the present disclosure comprises at least one first camera 6, configured for framing at least one portion of the support 3 from a first observation point. This configuration in the one represented in FIG. 1. Preferably, but non-limiting thereto, the at least one first camera 6 is arranged within the casing of the device 1. In an alternative embodiment, the first camera 1 is arranged outside the casing and is positioned in particular in a relative position with respect to the casing in correspondence thereof the casing is substantially transparent in order to allow the acquisition of one or more images of the sample of biological material 4.

In a preferred embodiment, the first observation point is fixed with respect to the frame of the device 1. In detail, the first camera 6 is arranged such as to acquire an image of at least one portion of the support 3 from a substantially vertical direction by framing the support 3 substantially in plan. In an embodiment, the first camera 6 is centered on a substantially vertical axis.

In particular, the first camera 6 is installed in a position such that it, in absence of the support 3, frames at least part of the supporting element 2, 2a, 2b. When the support 3 is positioned above the supporting element 2, 2a, 2b, part of the supporting element 2, 2a, 2b results covered by the support 3 therein leaned.

In an embodiment, the first camera 6 is configured for integrally framing the support 3, i.e. on the whole area thereof. In a further embodiment, the first camera 6 is configured for framing only a portion of the support 3, in particular only a central portion.

The first observation point lies at a height higher with respect to the height at which lies the supporting element 2, 2a, 2b. Equivalently, the first camera 6 frames, in use, the support 3 from the first side of the supporting element 2, 2a, 2b.

In an embodiment, the first camera 6 is a dot-matrix camera, for example and non-limiting thereto a CCD-type camera, configured for framing an image of an area of predefined shape, for example substantially rectangular or squared.

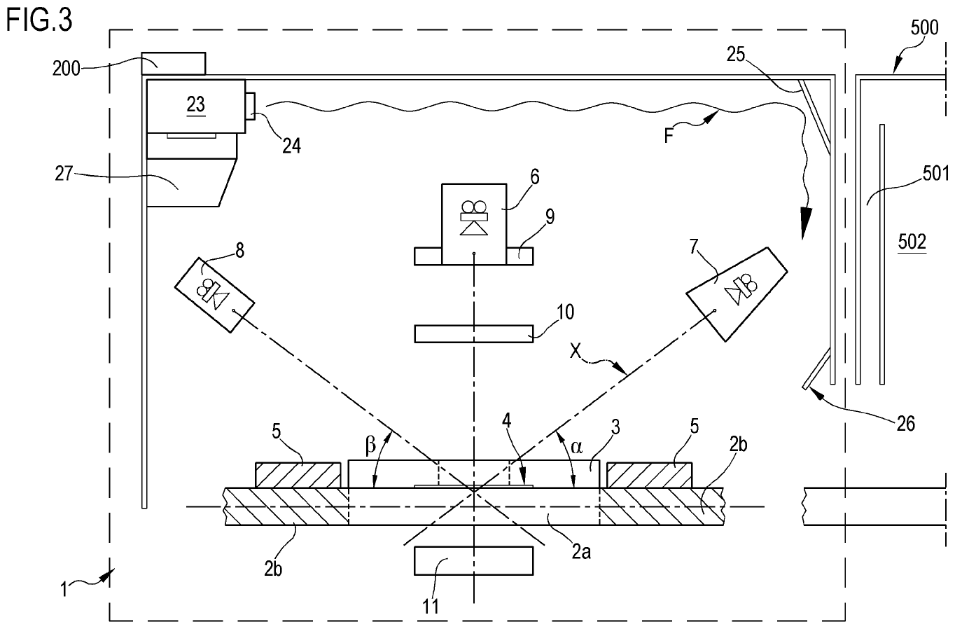

A further embodiment, non-limiting too, is characterized by the presence of two or more cameras. In FIG. 3 is represented an embodiment of the device 1 object of the present disclosure which comprises a second and a third camera. Further cameras, in particular those described below, can be arranged outside or within the casing. Embodiments are provided wherein part of the cameras is arranged within the casing and part of the cameras is arranged outside the casing.

The second camera 7 is configured for framing at least one portion of the support 3 from a second observation point. In a preferred embodiment, the second observation point is fixed with respect to the frame of the device 1. In detail, the second camera 7 is arranged such as to acquire an image of at least one portion of the support 3 from a direction oblique with respect to the support 3 and with respect to the vertical line of the support 3. The axis of framing of the second camera 7 is indicated with X, and forms a predefined first angle of framing predefined with respect to a substantially horizontal axis K. Preferably, but non-limiting thereto, the first angle of framing a is comprised between 10° and 80°, more preferably between 20° and 70°.

In particular, the second camera 7 is installed in a position such that, in absence of the support 3, it frames at least part of the supporting element 2, 2a, 2b.

In an embodiment, the second camera 7 is configured for integrally framing the support 3. In a further embodiment, the second camera 7 is configured for framing only a portion of the support 3, in particular only a central portion.

It is observed that in a specific embodiment the first camera 6 and the second camera 7 are positioned so as to frame only a portion of the support 3 (then, not integrally).

According to a preferred spatial arrangement and orientation that exists between the first camera 6 and the second camera 7, the portion of the support 3 framed by the first camera 6 is different (does not coincide) with respect to the portion of the support 3 framed by the second camera 7.

A particular spatial arrangement and orientation existing between the first camera 6 and the second camera 7 is such that the portion of the support 3 framed by the first camera 6 coincides at least partially, and optionally integrally, with the portion of the support 3 framed by the second camera 7. Therefore, the images acquired by them can be related to a same portion of the sample of biological material and/or of the support 3. A particular embodiment of the device 1 is such that the first camera 6 is linear (in particular, trilinear) and is configured for framing a portion of the support 3 corresponding to a radius thereof (if the support 3 has circular plan section) or half of its size (if the support 3 assumes a generic shape) in correspondence of the framing point, whereas the second camera 7 is linear (in particular, trilinear) and is configured for framing a portion of the support 3 corresponding to its diameter (if the support 3 has circular plan section) or the whole of a size thereof (if the support 3 has a generic shape) in correspondence of the framing point.

In a preferred embodiment, the optical installed on at least one between the first camera 6, the second camera 7 and the third camera 8 is characterized by a focal length different with respect to the others. The Applicant has conceived an embodiment of the device 1 wherein the first camera 6, the second camera 7 and the third camera 8 all have the same resolution, and in particular they are all trilinear cameras with resolution of 4096 pixel. Alternatively, the third camera 8 can have a lower resolution with respect to at least one between the first and the second camera. In an embodiment, the third camera is a trilinear camera with resolution of 1936 pixel. However, the first camera 6 and the second camera 7 have an optic with focus higher with respect to the third camera 8; the focus difference is such that with the third camera 8 it is possible to acquire an image of a portion of the support 3 that substantially comprises all the extension of the support 3 itself in correspondence of said portion, whereas with the first camera 6 and with the second camera 7 it is possible to acquire an image of a portion of the support 3 that comprises only part of the extension of the support 3 itself in correspondence of said portion.

If the support 3 is shaped with circular section, and supposing that the portion of the support 3 framed by the cameras is the substantially diametral one, this means that the first camera 6 and the second camera 7 will only be able to acquire an extension equal to the radius of said diametral portion, whereas third camera 8 will allow the framing of the entire diameter.

This technical characteristic allows to acquire, optionally simultaneously, one or more images of a portion of the support 3 with two densities of different graphic information; in the above example, in a case (first camera and second camera 7) the 4096 pixels will be used for acquiring an image of a radius, whereas in the other (third camera 8) for acquiring an image of a diameter.

Then, there is at least an embodiment of the device 1 wherein there are a first camera 6 and a second camera 7, respectively configured for framing a first and a second portion of the support 3, optionally at least partially coinciding.

The second observation point lies at a height higher with respect to the height at which lies the supporting element 2, 2a, 2b. Equivalently, the second camera 7 frames, in use, the support 3 from the first side of the supporting element 2, 2a, 2b.

In an embodiment the second camera 7 is a dot-matrix camera, for example and non-limiting thereto a CCD-type camera, configured for framing an image of an area of predefined shape, for example substantially rectangular or squared.

In a further non-limiting embodiment, at least one between the first and the second camera 6, 7, and in particular both the first camera 6 and the second camera 7 are linear cameras. A linear camera has sensors in array configured for acquiring a unique line of image, for example a line of a 1×1920 pixel or a line of 4096 pixel.

A particular embodiment of the device here described is such that the first and the second camera 6, 7 are of trilinear type; in this case the first camera 6 and the second camera 7 comprise, each one, three linear sensors. Preferably, each of the three linear sensors is configured for specifically receiving an optical radiation of a wavelength, more precisely of a window of wavelengths, distinct with respect to the wavelength, more precisely distinct with respect to the window of wavelengths, of the remaining sensors. This is made possible for example by dyes (types of pigment) present on the silica wafer upon which the sensor is realized. In a specific embodiment, a first of the three sensors is destined to receive the wavelengths of red (R), a second of the three sensors is destined to receive the wavelengths of green (G) and a third of the three sensors is destined to receive the wavelengths of blue (B). An image compensation system is present for compensating the space existing between a sensor and the other.

In other terms, at least one among the first camera 6, the second camera 7 and the third camera 8, when trilinear, comprises a set of three sensors of optical radiation each one configured for receiving optical radiations having frequency or wavelength lying in a receiving window substantially distinct with respect to the receiving window of the other sensors of the set of three.

The device 1 object of the present disclosure can also comprise a third camera 8, configured for framing at least one portion of the support 3 from a third observation point. In a preferred embodiment, the third observation point is fixed with respect to the frame of the device 1. In detail, the third camera 8 is arranged such as to acquire an image of at least one portion of the support 3 from a direction oblique with respect to the support 3 and with respect to the vertical line of the support 3. The framing axis of the third camera 8 is indicated with Z, and forms a predefined second angle of framing β with respect to a substantially horizontal axis K. Preferably, but non-limiting thereto, the second angle of framing β is comprised between 10° and 80°, more preferably between 20° and 70°.

In an embodiment, the first angle of framing and the second angle of framing are fixed, and are determined by a specific mounting configuration of the cameras on the frame of the device 1. However, the Applicant has conceived an embodiment wherein, even if not during the step of acquisition of the images, the position of at least one between the second camera 7 and the third camera 8, in particular the inclination of at least one between the second camera 7 and the third camera 8, can be adjusted—manually or automatically—on an angle comprised between the ranges [10°-80°] and more preferably [20°-70°].

In particular, the third camera 8 is installed in a position such that it, in absence of the support 3, frames at least part of the supporting element 2, 2a, 2b.

In an embodiment, the third camera 8 is configured for integrally framing the support 3.

The third observation point lies at a height higher with respect to the height at which lies the supporting element 2, 2a, 2b. Equivalently, the third camera 8 frames, in use, the support 3 from the first side of the supporting element 2, 2a, 2b.

In an embodiment, the third camera 8 is a dot-matrix camera, for example and non-limiting thereto a CCD-type camera, configured for acquiring an image of an area of predefined shape, for example substantially rectangular or squared. Alternatively, the third camera 8 is a linear camera (or trilinear); the characteristics for the linear or trilinear camera are above indicated, and therefore not repeated.

It is observed that in an embodiment, the first camera 6, the second camera 7 and the third camera 8 are activated in order to respectively acquire, at least a first, at least a second and at least a third image of a same portion of the sample of biological material and/or of the support 3.

In order to allow the acquisition of the image, the cameras here described clearly comprise a sensor. Optionally it is preferable that at least one between the first camera 6, the second camera 7 or the third camera 8 can be a camera with an active cooling system or device, operatively arranged in order to cause a cooling of the sensor of the camera; the active cooling device or system is configured for reducing said dark current, and can concur to optimize the quality of acquired images, in particular by reducing the noise of themselves.

A metal supporting frame, in particular but non-limiting thereto realized in aluminum, is provided for the purpose of supporting the first camera 6 and/or the second camera 7 and/or the third camera 8 in a predefined and stable position. The supporting frame for the camera is rigidly fixed to a frame on which it is installed the supporting element 2, 2a, 2b. Also the latter frame is realized in metallic material, preferably aluminum.

### Optical Radiation Sources

The device 1 object of the present disclosure comprises also a first optical radiation source 10 configured for lighting a portion of the support 3 from a first irradiation direction, in particular of lighting.

In a non-limiting embodiment, however, the first optical radiation source 10 is configured for lighting all the support 3. It shall then be intended that as “portion” of the support, it can be integrally considered also the support 3.

In a preferred but non-limiting embodiment the first optical radiation source 10 is used for the acquisition of the first image and of the second image respectively through the first camera 6 and the second camera 7.

Where the first optical radiation source 10 irradiates only a part of the support 3 (not integrally), the irradiated portion of the support 3 coincides with the portion of the support 3 framed by the first camera 6 and/or by the second camera 7.

Preferably, but in a non-limiting extent, the device 1 object of the present disclosure comprises a second optical radiation source 11, configured for lighting a portion of the support 3 from a second irradiation direction, in particular of lighting. In a non-limiting embodiment, however, the second optical radiation source 11 is configured for lighting all the support 3. It shall then be intended that as “portion” of the support, it can be integrally considered also the support 3.

Where the second optical radiation source 11 irradiates only a part of the support 3 (not integrally), the irradiated portion of the support 3 coincides with the portion of the support 3 framed by the first camera 6 and/or by the second camera 7.

As it is possible to observe for example from FIG. 1, the first optical radiation source 10 is arranged above the support 3 (and then above the supporting element 2, 2a, 2b) and lights this support with an irradiation direction, in particular of lighting that detects an acute angle with respect to the plane upon which lies the support 3 itself. The first optical radiation source lights, in use, the support 3 from first side of the supporting element 2, 2a, 2b.

The second optical radiation source 11 is arranged under the support 3 (and then under the supporting element 2, 2a, 2b) and lights this support with a direction that detects alternatively an acute angle with respect to the plane upon which lies the support 3 itself, or detects an angle substantially orthogonal with respect to the plane upon which lies the support 3. The second lighting source lights then the support 3 with a beam deriving from the second side of the supporting element 2, 2a, 2b and in detail passing, at least partially, by the first portion 2a of the supporting element itself.

In an embodiment, at least one between the first optical radiation source 10 and the second optical radiation source 11 is a source emitting an optical radiation in the visible domain.

For the purposes of the present disclosure, the visible domain comprises optical radiations the wavelengths thereof are substantially comprised in the range 400 nm-700 nm.

Preferably, but non-limiting thereto, the device 1 object of the present disclosure comprises a third optical radiation source 9, configured for lighting at least one portion of the support 3 from a third irradiation direction, in particular of lighting. The third irradiation direction, in particular of lighting is preferably inclined with respect to the first irradiation direction, in particular of lighting and to the second irradiation direction, in particular of lighting.

The third optical radiation source 9 is arranged above the support 3 (and then above the supporting element 2, 2a, 2b) and lights this support with an irradiation direction, in particular of lighting that detects an acute angle with respect to the plane upon which lies the support 3 itself. Alternatively, the irradiation direction, in particular of lighting is substantially orthogonal with respect to the plane upon which lies the support 3 itself. The third optical radiation source 9 can be a radiation source emitting an optical radiation in the visible domain.

The third optical radiation source 9 irradiates preferably a portion of the support 3 coinciding, or anyway geometrically superimposable, with the portion of the support 3 framed by the first camera 6 and/or by the second camera 7 and/or by the third camera 8.

In some embodiments, at least one optical radiation source selected among the first, the second and the third one, can be a radiating source in the infrared spectrum and/or in the ultraviolet spectrum.

For the purposes of the present disclosure, the infrared spectrum comprises radiations the wavelengths thereof are substantially comprised in the range 700 nm-1000 μm, and that in particular are comprised in the range of the infrared said “near” (700 nm-1.4 μm).

For the purposes of the present disclosure, the ultraviolet spectrum comprises radiations the wavelengths thereof are substantially comprised in the range 10 nm-400 nm.

Optionally, at least one among the first, the second and the third optical radiation source is an optical radiation source configured for being tunable in wavelength and in particular capable of emitting through the unique or combined action of one or more emitters, optical radiations in the range of the infrared and/or the visible and/or of the ultraviolet as above described.

The intensity of optical radiation of at least one among the first, the second and the third optical radiation source 10, 11, 9, can be manually and/or substantially automated adjusted, for example for facing different translucidity levels of the culture medium and/or according to an optical sensitivity of at least one of the cameras the device 1 can be provided therewith.

In detail, the third optical radiation source 9 is configured for substantially emitting a luminous line oriented toward the support 3 itself; in particular it is positioned so as to direct the luminous line along a direction substantially passing by the center of the support 3 itself. The third optical radiation source can be used, together with the associated camera, and in particular with the third camera, as a device destined to allow the three-dimensional processing of the image, in particular in order to detect a height variation of the sample of biological material. The height detection is carried out, among the other things, by assessing a luminosity difference acquired by the camera.

In a specific and non-limiting embodiment, the third optical radiation source 9 is a LASER source, and is in particular an optical radiation source that is temporally coherent and spatially coherent. The third optical radiation source 9 is a substantially strongly monochromatic optical radiation source. The bandwidth of the optical radiation of a LASER source is much lower with respect to the one of an incoherent optical source. In a specific embodiment, the LASER source emits substantially blue-violet light.

### The Conditioning of the Inner Space Defined by the Casing of the Device

As already partially described, the device 1 comprises a casing, housing therein said supporting element 2, 2a, 2b and, preferably, said at least one first camera 6. The casing, closeable, defines a first space 100 at least partially confined or delimited or closed.

As it will be better explained by the following portions of the description, the space 100 is configured for being conditioned in temperature and/or humidity by means of at least one air conditioning element 200, 101, 300, which in a first, non-limiting, embodiment, comprises an air conditioner.

This air conditioning element is destined, in use, to cause a variation and/or a maintenance of at least a predefined range of values of at least one between a temperature T of the air and humidity H of the air contained within the first space 100, and is preferably destined in use, to cause a variation and/or a maintenance of a predefined value of at least one between a temperature T of the air and humidity H of the air contained within the first space 100.

The purpose is to cause a reduction of, or to prevent the formation of, a condensation, in particular of a condensation of water vapour, on said at least one among said supporting element 2, 2a, 2b, said support 3 and said at least one first camera 6.

More in particular, in an embodiment, the space 100 is configured for being conditioned by means of an own air conditioner 200 of the device 1, preferably for creating a unique conditioned space with an incubation space of an incubator 500, wherein the humidity and/or temperature characteristics of the space are defined.

In an embodiment, the device 1 comprises a first air conditioner 200, preferably arranged within the casing or fixed to the casing. In an embodiment, the first air conditioner 200 can be provided with its own air filter.

The first air conditioner 200 is configured for conditioning the air contained within said first space 100 and is configured for causing a variation and/or a maintenance of at least a predefined range of values of at least one between a temperature T of the air and humidity H of the air contained within the first space 100.

Preferably, the first air conditioner 200 is configured for causing a variation and/or a maintenance of a predefined value of at least one between a temperature T of the air and humidity H of the air contained within the first space 100.

The purpose is to cause a reduction of, or to prevent the formation of, a condensation, in particular of a condensation of water vapour, on said at least one among said supporting element 2, 2a, 2b, said support 3 and said at least one first camera 6.

A further embodiment of the device 1 is similar to the one above described, and it also comprises at least one first camera 6. The casing, closeable, defines a first space 100 at least partially confined or delimited or closed.

Also in this new embodiment the space 100 is configured for being conditioned by means of an external conditioner, in particular the conditioner of the incubator 500, for creating a unique space the humidity and/or temperature characteristics thereof are defined.

In this case the casing of the device 1 comprises an inlet 101 for conditioned air configured for conditioning the air contained within the first space 100. The inlet 101 realizes a part of the air conditioning element 200, 101, 300 previously cited.

Tale inlet 101 is configured for causing a variation and/or a maintenance of at least a predefined range of values of at least one between a temperature T of the air and humidity H of the air contained within the first space 100.

More precisely, the inlet 101 is configured for causing a variation and/or a maintenance of a predefined value of at least one between a temperature T of the air and humidity H of the air contained within the first space 100.

The purpose is to cause a reduction of, or for preventing the formation of, a condensation, in particular of a condensation of water vapour, on said at least one among said supporting element 2, 2a, 2b, said support 3 and said at least one first camera 6.

In the light of the above description, it is therefore clear that the present disclosure describes as a whole a method for observing and acquiring images of samples of biological material 4.

This method first of all comprises a step of activation of at least one first air conditioner 200 of a device 1 for observing and for acquiring images of samples of biological material 4 and/or of activation of at least one second air conditioner 300 operatively connected to the device 1 by means of an inlet 101 for conditioned air arranged on a casing of the device 1, for conditioning the air contained in a first space 100, defined and at least partially confined or delimited or closed, by said casing.

The method herein described comprises, preferably subsequently, a step of arrangement of a support 3 for samples of biological material 4 on a supporting element 2, 2a, 2b housed within the casing of the device 1 for observing and for acquiring images of samples of biological material 4.

Subsequently, the method provides for a step of framing of at least one portion of the supporting element 2, 2a, 2b and/or, in use, of at least one predefined portion of the support 3, when housed on the supporting element 2, 2a, 2b, from a first point of observation, by means of at least one first camera 6 oriented toward the supporting element 2, 2a, 2b.

The step of framing follows the step of activation of the first air conditioner and/or of the second air conditioner 300, in order to have a lower risk of condensed water vapour on the support 3 and/or on the at least one first camera 6 and/or on the support 2, 2a, 2b.

In the method according to the present disclosure, the step of activation of the at least one first air conditioner 200 and/or of the at least one second air conditioner 300 determines a variation and/or a maintenance of at least a predefined range of values of at least one between a temperature T of the air and humidity H of the air contained within the first space 100 and, optionally, determines a variation and/or a maintenance of a predefined value of at least one between a temperature T of the air and humidity H of the air contained within the first space 100 and causes a reduction of, or prevents the formation of, a condensation, in particular of a condensation of water vapour, on said at least one among said supporting element 2, 2a, 2b, said support 3 and said at least one first camera 6.

The above is useful since in an embodiment the acquisition of at least one image of the sample of biological material 4 occurs with the support 3 in closed configuration, and in particular by activating the at least one first camera 6 in such a way that it frames the image of the sample of biological material 4 by observing through the cover of the support 3. If one between the upper wall or the lower wall of the cover included condensed water vapour, a correct image acquisition of the sample of biological material 4 would be compromised.

It is therefore clear that in a preferred embodiment the device 1 is configured for activating the at least one first camera 6 after the arrangement of the support 3 for samples of biological material 4 on the supporting element 2, 2a, 2b.

The device 1 is furthermore preferably configured for activating the at least one first camera 6 after an activation of the at least one first air conditioner 200 and/or of the second air conditioner 300.

The device 1 object of the present disclosure comprises a data processing unit 400. The technical features of the data processing unit 400 will be described in more detail below.

Alternatively, the device 1 object of the present disclosure is operatively connected to a data processing unit 400 operatively external to the device 1 and in particular operating in common both for the device 1 and for the incubator 500.

In this latter case the data processing unit 400 is operatively connected to the first air conditioner 200 of the device 1 and to the second air conditioner 300 of the incubator 500.

The data processing unit 400 is operatively connected with an incubator 500 configured and specifically destined to incubate samples of biological material 4. In particular the data processing unit 400 of the device 1 is operatively connected with a data processing unit 511 of the incubator 500. The data processing unit 511 can then be denominated “incubation data processing unit 511”, or “second data processing unit 511” (in this case the data processing unit described with the numerical reference 400 can be denominated “first data processing unit”).

The incubator 500, as it will be better explained in the following portion of the description, defines an incubation space 502 for the incubation of one or more samples of biological material 4.

In an embodiment, the incubator 500 is configured and specifically destined to be operatively coupled to the device 1 for receiving one or more supports 3 from a mover 5 of the device 1 or for supplying one or more supports 3 to the mover 5 of the device 1.

The data processing unit 400 is configured for receiving and/or transmitting, optionally automatically, air conditioning electronic data from the incubator 500 (in a specific embodiment from the incubation data processing unit 511 of the incubator 500 and to the incubation data processing unit 511, of the incubator 500).

In particular the conditioning electronic data are transmitted by a data processing unit 300 of the incubator 500. This data processing unit 300 is preferably operatively, in particular electrically connected, with the conditioner of the incubator 500.

Alternatively or in combination with the above, the data processing unit 400 is configured for receiving and/or transmitting, optionally automatically, air conditioning electronic data from the first air conditioner 200 and/or to said first air conditioner 200. This is in particular valid when the data processing unit 400 is a data processing unit common for the device 1 and for the incubator 500.

The data processing unit 400 is configured for setting a predefined range of values of at least one between a temperature T of the air and humidity H of the air contained within the first space 100, optionally said predefined value of at least one between a temperature T of the air and humidity H of the air contained within the first space 100, on the first and/or second air conditioner 200, 300, according to said electronic data, in such a way that the range of values of at least one between a temperature T of the air and humidity H of the air contained within the first space 100, optionally the predefined value of at least one between a temperature T of the air and humidity H of the air contained within the first space 100, is related or equal to range of values of at least one between a temperature T of the air and humidity H of the air contained within the incubation space 502 of the incubator 500, optionally related or equal to a predefined value of at least one between a temperature T of the air and humidity H of the air contained within the incubation space 502 of said incubator 500.

In a preferred but non-limiting embodiment the temperature range T of the air is a range of width equal to or lower than 5° C., or equal to or lower than 4° C., or equal to or lower than 3° C. or equal to or lower than 2° C. In a preferred but non-limiting embodiment, the range of values of humidity H of the air is a range of width equal to or lower than 5 percentage points, or equal to or lower than 4 percentage points, or equal to or lower than 3 percentage points, or equal to or lower than 2 percentage points.

It is observed that in the previous paragraphs the term “related” has been used. The predefined range of values of at least one between a temperature T of the air and humidity H of the air contained within the first space 100 is related to the range of values of at least one between a temperature T of the air and humidity H of the air contained within the incubation space 502 of the incubator 500 in such a way that:
at least one sample value of the predefined range of at least one between a temperature T of the air and humidity H of the air contained within the first space 100 is out of the range of values of at least one between a temperature T of the air and humidity H of the air contained within the incubation space 502 of the incubator 500, and/or
is in use maintained the reduction of, or is in use prevented the formation of, said condensation, in particular of said condensation of water vapour, on at least one between the supporting element 2, 2a, 2b, the support 3 and the at least one first camera 6.

Furthermore, said predefined value of at least one between a temperature T of the air and humidity H of the air contained within the first space 100 is related to the predefined value of at least one between a temperature T of the air and humidity H of the air contained within the incubation space 502 of the incubator 500 in such a way:
to lie at a predefined difference of temperature T of the air and/or humidity H of the air with respect to the at least one between a temperature T of the air and humidity H of the air contained within the incubation space 502 of the incubator 500, and/or
that is in use maintained the reduction of, or is in use prevented the formation of, said condensation, in particular of said condensation of water vapour, on at least one between the supporting element 2, 2a, 2b, the support 3 and the at least one first camera 6.

This difference of temperature T of the air can be equal to or lower than 5° C., or equal to or lower than 4° C., or equal to or lower than 3° C., or equal to or lower than 2° C.

### The Movement of the Sample of Biological Material

As already mentioned, the device 1 comprises at least one mover 5 configured for carrying at least one support 3 for samples of biological material 4 from a second space different with respect to the first space 100 toward the supporting element 2, 2a, 2b and/or vice versa, i.e. for transporting at least one support 3 for samples of biological material 4 from the first space 100 toward a second space different with respect to the first space 100.

Where the device 1 is effectively operatively coupled to an incubator 500, it is clear that the second space is an incubation space of the incubator 500.

The mover 5, which clearly comprises an end portion of mechanical gripping, can be actuated through a data processing unit of the device 1 by means of a servo-actuator that can be electric, or pneumatic, or hydraulic.

The mover 5 can include one or more pliers, even opposite, or one or more pushers.

In a non-limiting embodiment, the movement of the support 3 carried out through the mover 5 is carried out after the activation of at least one between the first air conditioner 200 or the second air conditioner 300. In an embodiment the movement of the support 3 carried out through the mover 5 is carried out after a stabilization of the temperature of the first space 100 and/or a synchronization of at least one between the temperature and/or the humidity of the air between the first space 100 and the incubation space.

### The Air Conditioner

The first air conditioner 200 is configured for generating a substantially free airflow, i.e. an airflow mainly non-channeled within the first space 100.

As can be seen in the attached figures, preferably the airflow F is a recirculating airflow, at least partially directed along a direction concordant with a direction of thermal convection within the first space 100.

The first air conditioner 200 and/or the second air conditioner 300 are thermal machines capable of decreasing or increasing a temperature and/or a humidity of a space within which they operate.

The first air conditioner 200, and/or the second air conditioner 300, comprises a compressor, a condenser, an evaporator, a laminating organ which corresponds to a throttling of a connection duct of the condenser, of the evaporator and of the compressor and/or can comprise a secondary heater having an electric resistor.

Each one between the first air conditioner 200 and the second air conditioner 300 can be configured for receiving an air-conditioning data signal destined to determine an increase of the temperature T of the air and/or an increase of the humidity H of the air and/or a reduction of the temperature T of the air and/or of the humidity H of the air.

Alternatively or in combination with the above, the first air conditioner 200 and/or the second air conditioner 300 can comprise an air heating unit, preferably electrically powered, and/or comprises an air-cooling unit, preferably electrically powered too.

The first air conditioner 200 and/or the second air conditioner 300 can have a device specifically configured and destined to determine an increase of the relative humidity of the air or a reduction of the relative humidity of the air.

### The Fan

The device 1 can comprise a fan 23 comprising an inlet and an outlet and configured for forcing, in correspondence of the outlet, an airflow within said first space 100.

The device 1 comprises also at least one guide 24, 25, 26 configured for guiding the airflow toward at least one between the supporting element 2, 2a, 2b, said support 3 and said at least one first camera 6.

In the non-limiting embodiment shown in the attached figures, the at least one guide 24, 25, 26 comprises at least one between the following elements:
a suction or thrust duct 24, positioned in substantial correspondence of the fan 23, and extending along at least a predefined direction which in the attached figures is a substantially horizontal direction and close to an upper limit of the volume of the first space 100;
at least one first deflector 25, preferably a first deflector 25 and a second deflector 26.

In a preferred but non-limiting embodiment, the first deflector is arranged substantially in an upper corner portion of the first space 100 and the second deflector is arranged in a substantially lower corner portion of the first space 100.

The first deflector 25 and the second deflector 26 are configured for determining a variation of an orientation of the airflow generated in use by the fan 23.

Preferably, the device 1 is specifically configured for determining a simultaneous activation of the at least one fan 23 with the first air conditioner 200 and/or with the second air conditioner 300 for causing said reduction of, or for preventing said formation of, a condensation, in particular of a condensation of water vapour, on said at least one among the supporting element 2, 2a, 2b, the support 3 and the at least one first camera 6.

The fan 23 can be preferably a pushing fan, and can have one or more impellers, and/or be a fan of tangential or axial type.

The fan 23 is electrically powered, and is in particular operatively, in particular electrically, connected to the data processing unit of the device 1.

The fan 23 in use is activated for generating an airflow, which in the attached figures is indicated by the letter “F”. The airflow F moves within the first space along a preferably predefined path, but when it exits from the fan outlet is substantially a free flow within the first space 100.

It is observed that, in a preferred embodiment, this airflow F is destined to contact with one among the first camera 6, and/or the second camera 7 and/or the third camera 8, in such a way that it is possible to prevent, or at least reduce, the risk of formation of excess humidity on delicate and even inner parts of the camera, for example actively cooled parts.

In an embodiment, the fan 23 is activatable or deactivatable independently from the first air conditioner 200 and/or by the second air conditioner 300.

Alternatively, the fan 23 can be activated after the activation of at least one between the first air conditioner 200 and/or the second air conditioner 300. This means that in an embodiment the fan 23 is a slave-type fan.

The fan 23, although described with a different reference number with respect to the reference number indicating the first air conditioner 200, it can be integrated within the first air conditioner 200; alternatively, it can be realized as a stand-alone element.

### The Incubator

A first embodiment of the incubator 500 is hereinafter described.

The incubator 500 comprises a casing which defines an incubation space 502 substantially insulated from the external space, and in particular at least partially, and preferably integrally confined or delimited or closed.

The casing of the incubator 500 comprises at least one door for the access to the incubation space 502; when the door is closed, said incubation space 502 is substantially insulated from the external space, and in particular is preferably integrally confined or delimited or closed.

Within the incubation space 502 is present at least one first incubation station 505. In the attached figures, it is shown an embodiment wherein are present a first incubation station 505 and a second incubation station 506 reciprocally side by side.

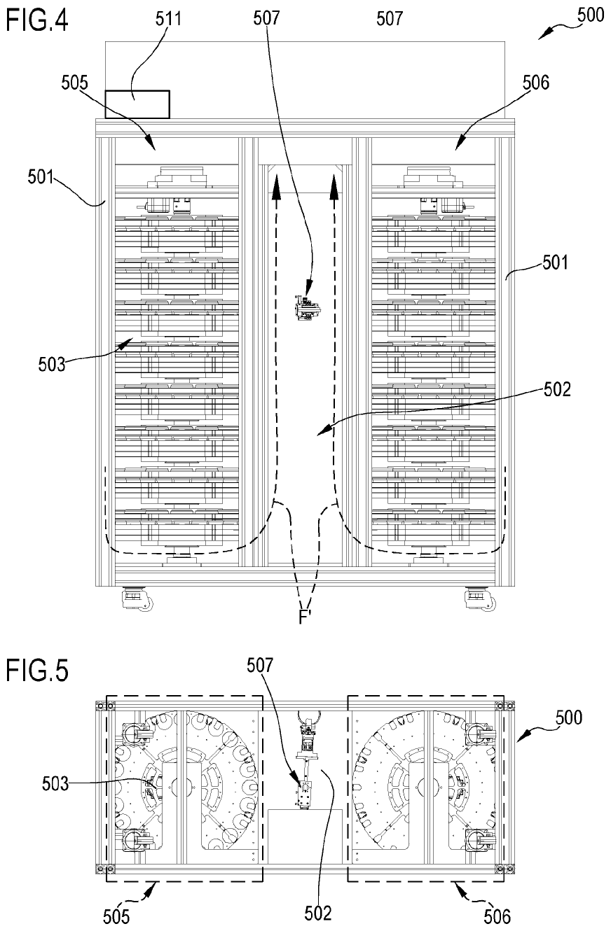

As schematically shown in FIG. 4 and in FIG. 5, in each incubation station is housed at least one support 3, and preferably are hosted a plurality of supports 3 arranged on movable supports 503 (or movable plates); the movable supports 503 are in particular fixed on a shaft rotating around a substantially vertical axis; such movable supports 503, alternatively or in combination, are movable on at least one between a substantially horizontal plane and a substantially vertical plane, and possibly movable with a composite rototranslation movement.

The incubator 500 comprises also an air conditioner 300, configured for conditioning the air contained within said incubation space 502 and configured for causing a variation and/or a maintenance of at least a predefined range of values of at least one between a temperature T of the air and humidity H of the air contained within the incubation space 502.

More in particular, the air conditioner 300 is configured for causing a variation and/or a maintenance of a predefined value of at least one between a temperature T of the air and humidity H of the air contained within said incubation space 502, for determining an incubation of at least one sample of biological material 4.

The incubator 500 is configured and specifically destined to be operatively coupled to a device 1 for observing and for acquiring images of samples of biological material 4, for receiving one or more supports 3 containing samples of biological material 4 from said device 1 and/or supplying one or more supports 3 containing samples of biological material 4 to said device 1.

In this embodiment, the incubator 500 is configured for transmitting toward the data processing unit of the device 1, and/or for receiving from the data processing unit of the device 1, a plurality of air conditioning electronic data. The incubator 500 is in this case destined to determine a synchronization of the operation of the first air conditioner 200, precisely of the device 1 or operatively associated to the device 1.

These electronic data are destined to determine a setting of said predefined range of values of at least one between a temperature T of the air and humidity H of the air contained within the first space 100, preferably a predefined value of at least one between a temperature T of the air and humidity H of the air contained within the first space 100, by action of the air conditioner 200 of the device 1 or operatively associated to the device 1, in such a way that the range of values of at least one between a temperature T of the air and humidity H of the air contained within the first space 100, and in particular the predefined value of at least one between a temperature T of the air and humidity H of the air contained within the first space 100, is related or equal to a range of values of at least one between a temperature T of the air and humidity H of the air contained within the incubation space 502 of the incubator 500, and is preferably related or equal to a predefined value of at least one between a temperature T of the air and humidity H of the air contained within the incubation space 502 of the incubator 500.

An alternative embodiment of the incubator 500 is hereinafter described.

The incubator 500 comprises a casing which defines an incubation space 502 substantially insulated from the external space, and in particular at least partially, and preferably integrally confined or delimited or closed.

The casing of the incubator 500 comprises at least one door for the access to the incubation space 502; when the door is closed, said incubation space 502 is substantially insulated from the external space, and in particular is preferably integrally confined or delimited or closed.

Within the incubation space 502 is present at least one first incubation station 505. In the attached figures, it is shown an embodiment wherein there are a first incubation station 505 and a second incubation station 506 reciprocally side by side.

In each incubation station is housed at least one support 3, and preferably are housed a plurality of supports 3 arranged on movable supports 503; the movable supports 503 are in particular fixed on a shaft rotating around a substantially vertical axis; such movable supports 503, alternatively or in combination, are movable on at least one between a substantially horizontal plane and a substantially vertical plane, and possibly movable with a composite rototranslation movement.

In this embodiment the incubator 500 does not comprise an its own air conditioner 300 but it is operatively connected with an air conditioner 200 of the device 1.

The incubator 500 comprises a handler 507, that in a preferred but non-limiting embodiment is arranged in a substantially central position of the casing, between the first incubation station 505 and the second incubation station 506. The handler 507 is configured and specifically destined to allow a handling of the supports of the samples of biological material. In a preferred but non-limiting embodiment, this handler 507 is conceived in order to realize in use a composite movement that can be rototranslatory along a horizontal plane and/or along a vertical plane.

The incubator 500 comprises at least one opening 510 of transit of supports 3, configured and specifically destined to make said incubation space 502 and the first space 100 for realizing an unique space having a same range of values of at least one between a temperature T of the air and humidity H of the air, and more in particular a same predefined value of at least one between a temperature T of the air and humidity H of the air, so that the unique space is at least partially confined or delimited or closed.

The incubator, preferably according to one of the two above-mentioned non-limiting embodiments, can be provided with an own fan, in use configured for being preferably operatively coupled with the second air conditioner 300.

The characteristics of the fan of the incubator 500 can be substantially similar to those of the fan 23 of the device 1, and therefore are not herein repeated.

In a specific embodiment, the incubator 500 comprises at least one fan configured for causing a bipartite airflow F′ within the incubation space 502.

In a preferred but non-limiting embodiment, the fan is configured for causing a first under-airflow F′ and a second under-airflow F′ which join together in a substantially central portion of the casing, wherein the manipulator 507 is preferably present. In a non-limiting embodiment the first under-airflow F′ and the second under-airflow F′ are upward flows in correspondence of the substantially central portion of the casing.

Preferably, the casing comprises two space walls, which are identified by the numerical reference 501. The space walls extend between a first portion positioned at a first height and a second portion positioned at a second height with respect to the first height. The first portion is preferably a bottom portion of the casing. The second portion is preferably a head portion of the casing, opposed with respect to the bottom portion. The space walls 501 preferably extend on the left side and on the right side, opposed with respect to the left side, of the casing 501. These left and right sides are arranged on a substantially orthogonal plane with respect to the plane on which are positioned the doors that allow the access to the incubation space 502.

In a preferred but non-limiting embodiment, within these space walls 501 the airflow F′ flows from top to bottom, and it is therefore a downward flow.

The Applicant has conceived further embodiments of the incubator 500, wherein only a unique space wall 501 is present, on the left side or alternatively on the right side, or still on a rear wall opposed with respect to the front wall of the casing.

At least in correspondence of the first portion, there is—for each space wall 501—a slit which allows the passage of the air from the space wall 501 to the incubation space 502.

Preferably, but in a non-limiting extent, the incubation space 502 is configured and specifically destined to have, in use, a temperature higher than the environmental one, typically in the range of 30-40° C., and/or particularly high relative humidity; the relative humidity can reach 90%, for instance. More generally, it is possible to state that an incubation space for samples 4 of biological material can have a temperature comprised between +5° C. and +45° C., preferably +10° C. and +40° C., and a humidity comprised between 30% and 100%, preferably between 40% and 100%, more preferably comprised between 50% and 95%.

It is then understood that the above-mentioned values of temperature T of the air and/or of humidity H of the air for the incubation space 502 can also be considered valid for the first space 100.

In an embodiment, the incubator 500 comprises a sensor of temperature of the air and/or of humidity of the air, configured for detecting the temperature of the air and/or the humidity of the air present within the incubation space 502. The sensor of temperature of the air and/or of humidity of the air is arranged within the incubation space 502. Preferably, the sensor of temperature of the air and/or of humidity of the air is operatively, preferably electrically, connected with the incubation data processing unit 511 of the incubator 500.

### Temperature Adjustment

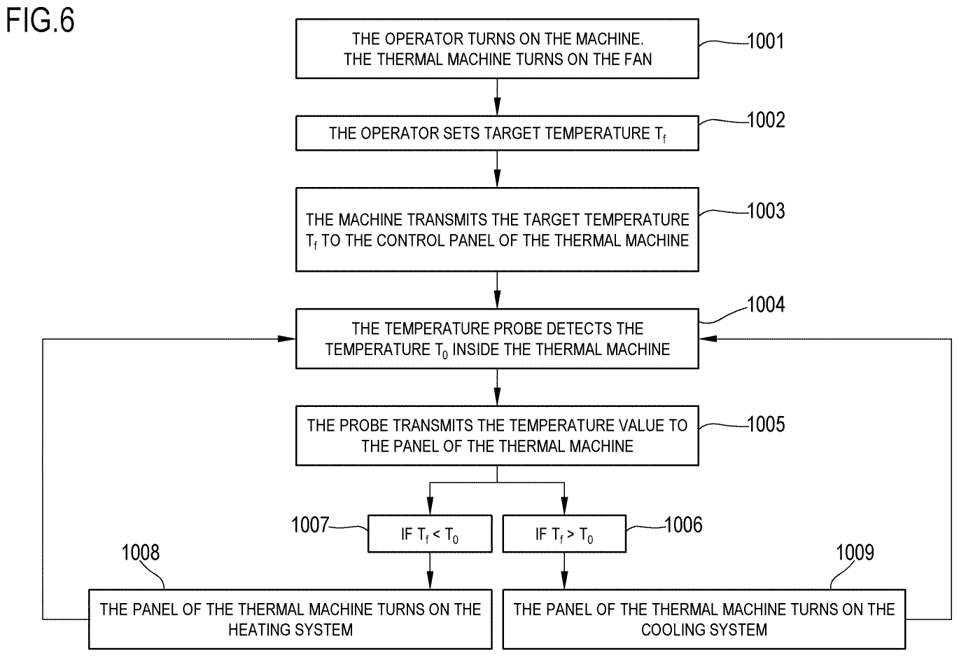

An operating cycle of temperature control for the first space 100 is here briefly described. FIG. 6 shows a flowchart that describes the operation of the adaptation of temperature made by the first air conditioner 200 of the device 1.

The block 1001 identifies a step wherein an operator switches on the device 1 (“machine”) and the fan 23 is activated in order to determine a recirculation of the air within of the first space 100.

It is observed that the fan 23 is activated by the air conditioner (defined in the block as “thermal machine”).

The block 1002 identifies a step following the step of the block 1001, wherein the operator sets a target temperature Tf to reach within the first space 100. The setting of the target temperature Tf occurs through a user interface of the device 1.

The block 1003 identifies a step following the step of the block 1002; in this step, it is transmitted the electronic data corresponding to the target temperature Tf to the control panel of the first air conditioner 200 (thermal machine).

The block 1004 identifies a step following the step of the block 1003; in this step, a temperature probe placed within the first space 100 detects a temperature To therein present and transmits an electronic data of temperature towards the control panel of the first air conditioner 200. The step of transmitting the electronic data of temperature towards the control panel of the first air conditioner 200 is represented by the block 1005.

The block 1006 and the block 1007 are two decisional blocks which identify two different conditions of a verification of whether the target temperature Tf is greater than the temperature To of the first space 100, or of whether the target temperature Tf is lower than the temperature To of the first space 100. In particular:
if Tf<To (block 1007), the panel of the first air conditioner 200 activates a heating system (block 1008) for increasing the temperature To of the first space 100 until the reaching of the target temperature Tf;
if Tf>=To (block 1006), the panel of the first air conditioner 200 activates a cooling system (block 1009) for decreasing the temperature To of the first space 100 until the target temperature Tf is reached.

The device 1 comprises a sensor of temperature of the air and/or of humidity of the air, configured for detecting the temperature of the air and/or of humidity of the air present within first space 100. The sensor of temperature of the air and/or of humidity of the air is arranged within the first space 100.

The sensor of temperature of the air and/or of humidity of the air is operatively connected to the first air conditioner 200 and/or to the second air conditioner 300, preferably but non-limiting thereto through the data processing unit of the device 1.

From the sensor of temperature of the air and/or of humidity of the air come data, preferably electronic data, indicative of a value of temperature of the air and/or of humidity of the air present within the first space 100.

In use, when the data transmitted by the sensor of temperature of the air and/or of humidity of the air indicate that the temperature of the air and/or the humidity of the air present within the first space 100 are compatible with the reduction, or absence, of condensation of water vapour, the first conditioner 200 and/or the second air conditioner 300 can be temporarily deactivated or controlled in order to cause the maintenance, preferably constant, of the temperature of the air and/or of the humidity of the air present within said first space 100. In particular, the above-described conditions occur when the electronic data provided by the sensor of temperature of the air and/or humidity of the air indicate a value of at least one between a temperature T of the air and humidity H of the air related to the predefined value of at least one between a temperature T of the air and humidity H of the air contained within the incubation space 502 of the incubator 500.

### General Data Related to Data Processing Units

The data processing units herein described with specific reference to the control of the air conditioner 200 of the device 1 and of the air conditioner 300 of the incubator 500 may comprise one or more among a general-purpose processor, an FPGA, one or more application-specific integrated circuits (ASIC), a programmable logic controller (PLC) or a dedicated data processor. On these data processing units can be executed data processing programs, stored on a non-transitory memory support.

Electronic data transmitted between the device 1 and the incubator 500, and in particular between the data processing units of the device 1 and the incubator 500 can be transmitted on a wired channel or on a wireless channel; such electronic data can be data of an electrical nature, transmitted on an electrically conductive cable, or data of an optical nature, transmitted on a wavelength range comprised between infrared, visible, and ultraviolet, on one or more optical fibers; such electronic data can be transmitted as a radio wave, on said wireless channel.

On the data processing unit can be executed a computer program which is stored on a memory support operatively joined to or disconnectable from the data processing unit itself. Such a memory support can for example—and non-limitingly—be a ROM memory or a small platter-type or solid-state Hard-Disk.

The computer program comprises software code portions that can be written in any programming language. The software code portions comprise instructions which, when executed, cause the execution of the steps of the method above described and, in a specific embodiment, at least the following steps.

A step that is actuated with the computer program herein described comprises an activation of at least one first air conditioner 200 of a device 1 for observing and for acquiring images of samples of biological material 4 and/or of activation of at least one second air conditioner 300 operatively connected to the device 1 by means of an inlet 101 for conditioned air arranged on a casing of the device 1, for conditioning the air contained in a first space 100, defined and at least partially confined or delimited or closed, by said casing. The data processing unit, through the computer program, sends to the first air conditioner 200 and/or to the second air conditioner 300 an activation command signal, in particular an electric signal, which determines the automated activation of the first air conditioner 200 and/or of the second air conditioner 300.

A subsequent step actuated with the computer program herein described comprises an activation of at least one mover for carrying out an arrangement of a support 3 for samples of biological material 4 on a supporting element 2, 2a, 2b housed within the casing of the device 1 for observing and for acquiring images of samples of biological material 4.

The computer program comprises portions of software code that when executed determine the activation at least of the first camera 6 in such a way that it is possible to realize a framing of at least one portion of the supporting element 2, 2a, 2b and/or, in use, of at least one predefined portion of the support 3, when housed on the supporting element 2, 2a, 2b, from a first point of observation.

The step of framing follows the step of activation of the first air conditioner 200 and/or of the second air conditioner 300, in order to have a lower risk of condensed water vapour on the support 3 and/or on the at least one first camera 6 and/or on the support 2, 2a, 2b.

The invention is not limited to the embodiments shown in the attached figures. For this reason, reference numbers in the following claims are provided for the sole purpose of increasing the intelligibility thereof and shall not be intended as limiting.

It is then clear that to the object of the present disclosure can be applied additions, modifications or variants obvious for an expert in the art without exiting from the scope of the attached claims.

2. Device according to claim 1, comprising at least one mover configured to carry at least one support for samples of biological material from a second space different with respect to said first space toward said supporting element and/or vice versa; said second space being an incubation space of an incubator configured to incubate samples of biological material contained in one or more supports, said incubator being configured to be operatively coupled to said device for receiving one or more supports from the mover of the device, wherein said support is a closable support, having at least an open configuration and a closed configuration, wherein in said closed configuration the support delimits a predefined inner volume, the support comprising at least one upper wall, a lower wall opposed with respect to said upper wall and at least one lateral wall joined to said lower wall, and wherein said support comprises a cover and at least part of the upper wall is a wall of said cover, and wherein the sample of biological material is contained within said predefined inner volume and on said lower wall,
and wherein said device is configured to handle said support in said closed configuration and/or is configured to acquire at least one image of the sample of biological material with said support in said closed configuration.

3. Device according to claim 1, comprising, or operatively connected with, a data processing unit operatively connected with an incubator configured to incubate samples of biological material contained in one or more supports, within an incubation space at least partially confined or delimited or closed, said incubator being configured to be operatively coupled to said device for receiving and/or supplying one or more supports from/to a mover of the device,
the data processing unit being configured to receive and/or transmit air conditioning electronic data from the incubator and/or toward the incubator, or being configured to receive and/or transmit air conditioning electronic data from the incubator and/or toward the incubator and from said first air conditioner and/or to said first air conditioner, and for setting said predefined range of values of at least one between a temperature of the air and humidity of the air contained within said first space, on said first and/or second air conditioner, according to said electronic data, in such a way that said range of values of at least one between a temperature of the air and humidity of the air contained within said first space is related or equal to range of values of at least one between a temperature of the air and humidity of the air contained within the incubation space of said incubator;
wherein the data processing unit is configured to automatically receive, air conditioning electronic data from an incubation data processing unit, of said incubator.

4. Device according to claim 1, wherein at least one between said first air conditioner and said second air conditioner is configured to generate a free airflow within said first space and directed at least partially along a direction concordant with a direction of thermal convection within said first space.

5. Device according to claim 1, comprising:
at least one fan comprising an inlet and an outlet and configured to force, in correspondence of said outlet, an airflow within said first space;
at least one guide configured to guide said airflow toward at least one between said supporting element, said support and said at least one first camera;
the device being specifically configured to determine a simultaneous activation of said at least one fan with said first air conditioner and/or with said second air conditioner to cause said reduction of, or to prevent said formation of, a condensation, in particular of a condensation of water vapour, on said at least one among said supporting element, said support and said at least one first camera.

6. Device according to claim 5, wherein:
said inlet of said at least one fan is configured to suck a part of the air present within said first space in correspondence of a first portion of said first space and the outlet of said fan is configured to blow air in correspondence of a second portion of said first space different with respect to the first portion of said first space; and/or
said fan is arranged in at least one predetermined portion within said casing and/or in said first space;
at least one between said first portion and said second portion is a substantially upper or top portion of said first space.

7. Device according to claim 5, wherein said at least one guide comprises at least one between the following elements:
a suction or thrust duct, positioned in substantial correspondence of said at least one fan, and extending along at least a predefined direction; and
at least one first deflector;
said at least one guide being configured to determine a variation of an orientation of the airflow generated in use by said fan, and wherein said at least one guide and said at least one fan are configured and arranged in a reciprocal spatial orientation to determine a substantially closed recirculation of air between said inlet and said outlet.

8. Device according to claim 6, wherein said at least one first deflector is positioned within said casing and in said substantially upper or top portion of said first space,
and/or wherein said second deflector is positioned within said casing and in a substantially lower or bottom portion of said first space;
wherein said casing comprises an upper wall, a lower wall opposed with respect to the upper wall and at least one lateral wall having a first and a second portion, the first portion being joined to the upper wall, the second portion being joined to the lower wall,
wherein at least one between said first and said second portion are ending portions,
wherein said casing defines at least one first corner portion in correspondence of a zone adjacent to the union between the upper wall and the lateral wall and/or adjacent to said first portion and defines at least one second corner portion in correspondence of a zone adjacent to the union between the lower wall and the lateral wall and/or adjacent to said second portion,
and wherein said first deflector is positioned in said first corner portion and/or wherein said second deflector is positioned in said second corner portion.

9. Device according to claim 1, comprising at least one first optical radiation source configured to irradiate at least said predefined portion of the support from a first irradiation direction, in such a way that, in use, at least one portion of the sample of biological material is irradiated through the optical radiation of the first optical radiation source;
and wherein said first or second air conditioner is configured to cause a reduction of, or for preventing the formation of, a condensation, in particular of a condensation of water vapour, on said at least one among said supporting element, said support and said at least one first optical radiation source.

10. Device according to claim 1, comprising a filter for the air contained in said first space; the filter is connected on the inlet of said fan and/or to at least one between said first air conditioner and said second air conditioner, and is configured to filter the air before it arrives on the outlet of said fan;
the filter being a filter comprising at least one layer, and being a filter destined to filter volatile organic compounds, dusts and bacteria or viruses, and/or being an electrostatic filter.

11. Method for observing and for acquiring images of samples of biological material, comprising:
a step of activation of at least one first air conditioner of a device for observing and for acquiring images of samples of biological material and/or of activation of at least one second air conditioner operatively connected to the device by means of an inlet for conditioned air arranged on a casing of the device, for conditioning the air contained in a first space, defined and at least partially confined or delimited or closed, by said casing;
a step of arrangement of a support for samples of biological material on a supporting element housed within the casing of the device for observing and for acquiring images of samples of biological material;
a step of framing of at least one portion of the supporting element and/or, in use, of at least one predefined portion of the support, when housed on the supporting element, from a first point of observation, by means of at least one first camera oriented toward the supporting element;
wherein the step of activation of said at least one first air conditioner and/or of said at least one second air conditioner determines a variation and/or a maintenance of at least a predefined range of values of at least one between a temperature of the air and humidity of the air contained within said first space and determines a variation and/or a maintenance of a predefined value of at least one between a temperature of the air and humidity of the air contained within said first space and causes a reduction of, or prevents the formation of, a condensation, in particular of a condensation of water vapour, on said at least one among said supporting element, said support and said at least one first camera.

12. Method according to claim 11, comprising a step of transportation of at least one support for samples of biological material from a second space different with respect to said first space toward said supporting element and/or vice versa, the step of transportation occurring through a mover of the device, wherein said second space is an incubation space of an incubator configured and specifically destined to incubate samples of biological material contained in one or more supports, and wherein the incubator is configured to be operatively coupled to said device for receiving one or more supports from the mover of the device,
wherein said support is a closable support, having at least an open configuration and a closed configuration, wherein in said closed configuration the support delimits a predefined inner volume, the support comprising at least one upper wall, a lower wall opposed with respect to said upper wall and a lateral wall joined to said lower wall, and wherein said support comprises a cover and at least part of the upper wall is a wall of said cover, and wherein the sample of biological material is contained within said predefined inner volume and on said lower wall,
the method comprising a handling of the support in said closed configuration, and/or comprising a step of acquisition of at least one image of said sample of biological material with said support in said closed configuration.

13. Method according to claim 11, comprising a step of activation of a data processing unit of the device, or operatively external to the device, the data processing unit being operatively connected with an incubator configured to incubate samples of biological material within an incubation space of the incubator at least partially confined or delimited or closed, contained in one or more supports, said incubator being configured to be operatively coupled to said device to receive one or more supports from a mover of the device,
the method comprising a step of reception and/or transmission, through said data processing unit, of air conditioning electronic data from the incubator and/or to the incubator and/or comprising a step of reception and/or transmission, through said data processing unit, of air conditioning electronic data from the first air conditioner and/or to said first air conditioner;
the method comprising a step of setting of said predefined range of values of at least one between a temperature of the air and humidity of the air contained within said first space on said first and/or second air conditioner, according to said electronic data, in such a way that said range of values of at least one between a temperature of the air and humidity of the air contained within said first space is related or equal to range of values of at least one between a temperature of the air and humidity of the air contained within the incubation space of said incubator;
wherein the method comprises a step of automatic reception, through the data processing unit, of air conditioning electronic data from an incubation data processing unit, of said incubator.

14. Method according to claim 11, wherein the step of activation of said first air conditioner and/or of said second air conditioner determines the generation of a free airflow and at least by effect of the activation of at least one between said first and said second air conditioner, said free airflow is directed at least partially along a direction concordant with a direction of thermal convection within said first space (100).

15. Method according to claim 11, comprising:
a step of activation of at least one fan of said device and comprising an inlet and an outlet, for forcing, in correspondence of said outlet, an airflow within said first space;
a step of guide, through at least one guide of said device, of said airflow toward at least one between said supporting element, said support and said at least one first camera;
the method comprising a simultaneous execution of the step of activation of said fan with the step of activation of said first air conditioner and/or of said second air conditioner to cause said reduction of, or to prevent the formation of, a condensation, in particular of a condensation of water vapour, on said at least one among said supporting element, said support and said at least one first camera.

16. Method according to claim 15, wherein the step of activation of said at least one fan determines a suction, through the inlet, of a part of the air present within said first space from a first portion of said first space and determines a blowing, through said outlet, of air in correspondence of a second portion of said first space different with respect to the first portion of said first space;
and/or wherein the method comprises arranging the fan in at least a predetermined position, within said casing and/or in said first space;
wherein at least one between said first portion and said second portion is a substantially upper or top portion of said first space.

17. Method according to claim 15, wherein said at least one guide comprises at least one between the following elements:
a suction or thrust duct, positioned in substantial correspondence of said at least one fan, and extending along at least a predefined direction; and
at least one first deflector;
and wherein the step of guiding, through at least one guide of said device, of said airflow, comprises a variation of an orientation of the airflow generated through the step of activation of said fan,
the method comprising a substantially closed recirculation of air between said inlet and said outlet, said recirculation being determined by a reciprocal spatial orientation between said at least one guide and said at least one fan.

18. Method according to claim 16, comprising a step of positioning of the at least one first deflector within said casing and in said substantially upper or top portion of said first space,
and/or comprising a step of positioning of the second deflector within said casing and in a substantially lower or bottom portion of said first space;
said casing comprising an upper wall, a lower wall opposed with respect to the upper wall and at least one lateral wall having a first and a second portion, the first portion being joined to the upper wall, the second portion being joined to the lower wall,
wherein at least one between said first and said second portion are ending portions,
wherein said casing defines at least one first corner portion in correspondence of a zone adjacent to the union between the upper wall and the lateral wall and/or adjacent to said first portion and defines at least one second corner portion in correspondence of a zone adjacent to the union between the lower wall and the lateral wall and/or adjacent to said second portion,
and wherein the step of positioning of said first deflector determines an allocation of said first deflector in said first corner position and/or wherein the step of positioning of said second deflector determines an allocation of said second deflector in said second corner portion.

19. Method according to claim 11, comprising a step of activation of at least one first optical radiation source to irradiate at least said predefined portion of the support from a first irradiation direction, in such a way that, in use, at least one portion of the sample of biological material is irradiated through the optical radiation of the first optical radiation source;
wherein the step of activation of said at least one first air conditioner of said device and/or the step of activation of said at least one second air conditioner causes a reduction of, or prevents the formation of, a condensation, in particular of a condensation of water vapour, on said at least one among said supporting element, said support and said at least one first optical radiation source.

20. Method according to claim 11, comprising a step of filtering of the air contained in said first space by means of a filter, wherein the filter is connected on the inlet of said fan and/or to at least one between said first air conditioner and said second air conditioner and is configured to carry out a filtering of the air before it arrives on the outlet of said fan.

21.-25. (canceled)
